SEQUENCE LISTING (22 sequences)

SEQ ID NO: 1 - DNA, 19 bp, synthetic construct
  source 1..19: mol_type = other DNA; note = mCMTM6_shRNA_1 sequence
tgcctaacagaaagcgtgt

SEQ ID NO: 2 - DNA, 21 bp, synthetic construct
  source 1..21: mol_type = other DNA; note = mCMTM6_shRNA_2 sequence
ccggagatttaatgagtgttt

SEQ ID NO: 3 - DNA, 21 bp, synthetic construct
  source 1..21: mol_type = other DNA; note = mCMTM6_shRNA_3 sequence
cctcagctgaaattgctgcaa

SEQ ID NO: 4 - DNA, 18 bp, synthetic construct
  source 1..18: mol_type = other DNA; note = hCMTM6_shRNA_1 sequence
ccggcctttcttctgagt

SEQ ID NO: 5 - DNA, 17 bp, synthetic construct
  source 1..17: mol_type = other DNA; note = hCMTM6_shRNA_2 sequence
ccggctttcttctgagt

SEQ ID NO: 6 - DNA, 21 bp, synthetic construct
  source 1..21: mol_type = other DNA; note = hCMTM6_shRNA_3 sequence
cctttcttctgagtctcctta

SEQ ID NO: 7 - DNA, 50 bp, synthetic construct
  source 1..50: mol_type = other DNA; note = mCMTM6_shRNA_4 sequence
tgcctaacagaaagcgtgtctcgagacacgctttctgttaggcatttttg

SEQ ID NO: 8 - DNA, 54 bp, synthetic construct
  source 1..54: mol_type = other DNA; note = mCMTM6_shRNA_5 sequence
ccggagatttaatgagtgtttctcgagaaacactcattaaatctccggtttttg

SEQ ID NO: 9 - DNA, 54 bp, synthetic construct
  source 1..54: mol_type = other DNA; note = mCMTM6_shRNA_6 sequence
cctcagctgaaattgctgcaactcgagttgcagcaatttcagctgaggtttttg

SEQ ID NO: 10 - DNA, 58 bp, synthetic construct
  source 1..58: mol_type = other DNA; note = hCMTM6_shRNA_4 sequence
ccggcctttcttctgagtctccttactcgagtaaggagactcagaagaaaggtttttg

SEQ ID NO: 11 - DNA, 59 bp, synthetic construct
  source 1..59: mol_type = other DNA; note = hCMTM6_shRNA_5 sequence
ccggctttcttctgagtctccttatctcgagataaggagactcagaagaaagttttttg

SEQ ID NO: 12 - DNA, 55 bp, synthetic construct
  source 1..55: mol_type = other DNA; note = hCMTM6_shRNA_6 sequence
cctttcttctgagtctccttactcgagtaaggagactcagaagaaaggttttttg

SEQ ID NO: 13 - DNA, 20 bp, synthetic construct
  source 1..20: mol_type = other DNA; note = mCMTM6_sgRNA sequence
cctggccgcctacttcgtcc

SEQ ID NO: 14 - DNA, 20 bp, synthetic construct
  source 1..20: mol_type = other DNA; note = hCMTM6_sgRNA_1 sequence
ccgggtcctcctccgtagtg

SEQ ID NO: 15 - DNA, 20 bp, synthetic construct
  source 1..20: mol_type = other DNA; note = hCMTM6_sgRNA_2 sequence
tcacaatgtactttatgtgg

SEQ ID NO: 16 - DNA, 21 bp, synthetic construct
  source 1..21: mol_type = other DNA; note = mPD-L1_shRNA sequence
ccgaaatgatacacaattcga

SEQ ID NO: 17 - DNA, 25 bp, synthetic construct
  source 1..25: mol_type = other DNA; note = hPD-L1_shRNA_1 sequence
ccggcgaattactgtgaaagtcaat

SEQ ID NO: 18 - DNA, 25 bp, synthetic construct
  source 1..25: mol_type = other DNA; note = hPD-L1_shRNA_2 sequence
ccggctgacattcatcttccgttta

SEQ ID NO: 19 - DNA, 58 bp, synthetic construct
  source 1..58: mol_type = other DNA; note = hPD-L1_shRNA_1 sequence
ccggcgaattactgtgaaagtcaatctcgagattgactttcacagtaattcgtttttg

SEQ ID NO: 20 - DNA, 62 bp, synthetic construct
  source 1..62: mol_type = other DNA; note = hPD-L1_shRNA_2 sequence
ccggctgacattcatcttccgtttactcgagtaaacggaagatgaatgtcagccggtttttg

SEQ ID NO: 21 - protein, 9 aa, synthetic construct
  source 1..9: note = RGD polypeptide
CDCRGDCFC

SEQ ID NO: 22 - protein, 4 aa, synthetic construct
  source 1..4: note = tag sequence
GGGS

(19) United States
(12) Patent Application Publication
(10) Pub. No.: US 2025/0223599 A1
(43) Pub. Date: Jul. 10, 2025

(54) PREPARATION AND ANTI-TUMOR APPLICATION OF GENE THERAPY VECTOR INTERFERING CKLF-LIKE MARVEL TRANSMEMBRANE DOMAIN-CONTAINING PROTEIN 6 (CMTM6) EXPRESSION

(71) Applicant: SHANGHAI INSTITUTE OF MATERIA MEDICA, CHINESE ACADEMY OF SCIENCES, Shanghai (CN)

(72) Inventors: Likun GONG, Shanghai (CN); Yiru LONG, Shanghai (CN); Jianhua SUN, Shanghai (CN); Runqiu CHEN, Shanghai (CN); Xiaolu YU, Shanghai (CN); Yongliang TONG, Shanghai (CN); Qiuping QIN, Shanghai (CN); Tingting LIU, Shanghai (CN); Junjiu XU, Shanghai (CN)

(21) Appl. No.: 18/850,967

(22) PCT Filed: Mar. 24, 2023
(86) PCT No.: PCT/CN2023/083888

§ 371 Date: Sep. 25, 2024

(30) Foreign Application Priority Data
Mar. 25, 2022 (CN) 202210307613.4

(51) Int. Cl. C12N 15/113; A61K 45/06; A61P 35/00; C12N 15/11; C12N 15/86

(52) U.S. Cl. CPC C12N 15/1138; A61K 45/06; A61P 35/00; C12N 15/111; C12N 15/86; C12N 2310/14; C12N 2310/351; C12N 2310/531; C12N 2740/15043; C12N 2750/14143

(57) ABSTRACT

Disclosed are preparation and anti-tumor application of a gene therapy vector interfering CKLF-like MARVEL transmembrane domain-containing protein 6 (CMTM6) expression. Specifically, disclosed are a gene therapy vector encoding a gene sequence targeting CMTM6 and a derivative thereof, a gene sequence encoded by a vector, a preparation method, and a use of a vector alone and in combination with other drugs in treating tumors. These gene therapy vectors comprise adeno-associated viruses and lentiviruses, etc., can inhibit the expression of CMTM6 in tumor tissues, can effectively inhibit the in-vivo growth of mouse and human colorectal cancer, melanoma, liver cancer, breast cancer, non-small cell lung cancer and the like by improving the tumor immunosuppression microenvironment, exhibit a strong anti-tumor effect in combination with immune checkpoint antibodies, chemotherapeutic drugs, immunoagonistic drugs, and metabolic regulation drugs, and have significant efficacy on PD-L1-deficient tumors and immune checkpoint antibody drug-resistant tumors.

### TECHNICAL FIELD

The present invention relates to the field of biomedicine, specifically to the preparation and application of gene therapy vectors that interfere with the expression of CKLF-like MARVEL transmembrane domain-containing protein 6 (CMTM6), particularly for the use of adeno-associated virus and lentivirus gene drugs targeting CMTM6 in the treatment of tumors.

### BACKGROUND

The development of immune checkpoint blockade therapy shows two obvious trends: the discovery of novel immune checkpoint molecules and the combination therapy. In addition to the classic CTLA-4 and PD-1/PD-L1, novel immune checkpoint molecules such as LAG3, TIM3, TIGIT, and Siglec-15 have been progressively discovered and applied, broadening our understanding of T-cell activity regulation and expanding the range of target options for tumor immunotherapy. However, the efficacy and response rate of tumor immunotherapy, represented by immune checkpoint blockade therapy, are still relatively low. Exploring new therapeutic targets and combination therapy regimens remain one of the effective ways to improve therapeutic effects.

CKLF-like MARVEL transmembrane domain-containing protein 6 (CMTM6) is a member of the human CKLF-like MARVEL transmembrane domain-containing family (CMTM family). CMTM6 contains a four-transmembrane MARVEL domain, and there is limited research on the biological functions of both CMTM6 and proteins belonging to this family. On the surface of tumor cells, CMTM6 can act as a post-translational modification regulatory molecule for PD-L1, interact with PD-L1 and maintain the expression level of PD-L1 on the cell membrane by inhibiting its ubiquitin-proteasome degradation pathway and endosome-lysosome degradation pathway. CMTM6 is a potential target for tumor immunotherapy, as well as a biomarker for tumor diagnosis and prognosis.

Gene therapy is a means of introducing target genes into target cells to achieve therapeutic goals. The existing gene therapy vectors comprise liposomes, nanocarriers, naked DNAs, adenovirus vectors, retrovirus vectors, lentivirus vectors, and adeno-associated virus vectors, etc.

At present, the development of tumor immunotherapy based on gene therapy is still in its infancy: on the one hand, the effectiveness of existing tumor immune gene therapy remains unsatisfactory, and the efficacy of preclinical and clinical trials needs to be improved; on the other hand, most of the targets selected for existing tumor immune gene therapy are commonly used immune checkpoint molecules or cytokines, etc. These targets often have antibody drugs or recombinant protein drugs developed already, while gene therapies targeting these targets have not shown significant advantages. Therefore, tumor immune gene therapy requires both improving treatment efficacy and response rate, as well as exploring targets that pose challenges for traditional antibody and recombinant protein development. Both of these require the application of novel tumor immune therapy targets in gene therapy and the exploration of combination therapy to enhance treatment effectiveness.

As a potential target for tumor immunotherapy, the unique structural characteristics of CMTM6 as a membrane protein determine the difficulty in developing other types of drugs, yet it can be effectively utilized through gene therapy vectors. Therefore, the development of a novel gene therapy vector that interferes with CMTM6 expression, achieving satisfactory tumor therapeutic effects and enabling widespread application in the field of anti-tumor therapy, can enhance the approaches and prospects of gene therapy in the realm of tumor immunotherapy.

### SUMMARY OF THE INVENTION

The purpose of the present invention is to provide a novel cancer treatment target and corresponding treatment methods targeting the novel target.

Another purpose of the present invention is to provide a gene therapy vector that interferes with CMTM6 expression, along with its derivatives and compositions, which exhibit anti-tumor effects.

In the first aspect of the present application, it provides a use of a gene therapy vector for targeted downregulation of CMTM6, in the manufacture of a composition or formulation, wherein the composition or formulation is used for: (a) prevention and/or treatment of tumors; and/or (b) inhibition of tumor cells.

In another preferred embodiment, the prevention and/or treatment by the gene therapy vector comprises inhibiting the growth and/or metastasis of the tumors.

In another preferred embodiment, the inhibition by the gene therapy vector comprises inhibiting the growth and/or metastasis of the tumor cells.

In another preferred embodiment, the gene therapy vector is used in combination with a drug selected from the group consisting of: an immune checkpoint antibody, an immune agonist, a chemotherapy drug, a lipid metabolism regulating drug, a glucose metabolism regulating drug, an additional gene therapy vector, and a combination thereof.

In another preferred embodiment, the combination therapy regimen involves administering the gene therapy vector in combination with the chemotherapy drug and/or lipid metabolism regulating drug.

In another preferred embodiment, the tumors are tumors of mammals (including humans and non-human mammals).

In another preferred embodiment, the non-human mammals are mice.

In another preferred embodiment, the tumors are human-derived tumors.

In another preferred embodiment, the tumors are those that have shown ineffectiveness or failure for treatment with immune checkpoint antibodies or immune checkpoint inhibitors, or those that are unsuitable for treatment with immune checkpoint antibodies or immune checkpoint inhibitors.

In another preferred embodiment, the tumors are those that have shown ineffectiveness or failure for treatment with PD-L1 antibodies or PD-L1 inhibitors, or those that are unsuitable for treatment with PD-L1 antibodies or PD-L1 inhibitors.

In another preferred embodiment, the tumors or tumor cells are tumors or tumor cells with high expression of CMTM6.

In another preferred embodiment, the immune checkpoint is selected from the group consisting of: PD-1, PD-L1, CTLA-4, B7-H3, LAG-3, VISTA, CD47, TIM-3, TIGIT, BTLA, Siglec-15, etc.

In another preferred embodiment, the immune checkpoint antibodies are PD-L1 antibodies and CTLA-4 antibodies.

In another preferred embodiment, the additional gene therapy vector refers to a gene therapy vector targeting the immune checkpoint.

In another preferred embodiment, the tumors are tumors with expression of PD-L1 and tumors with no expression of PD-L1.

In another preferred embodiment, the tumors are selected from the group consisting of: tumors with high expression of PD-L1, tumors with medium expression of PD-L1, and tumors with low expression of PD-L1.

In another preferred embodiment, the tumors are tumors with medium expression of PD-L1 or tumors with low expression of PD-L1.

In another preferred embodiment, the tumors are tumors with low expression of PD-L1.

In another preferred embodiment, “high expression of PD-L1” refers to a ratio (E1/E0)>1, preferably ≥1.5, and more preferably ≥2.0, wherein E1 is the amount of PD-L1 expressed by the tumor and E0 is the amount of PD-L1 expressed by a normal tumor.

In another preferred embodiment, “medium expression of PD-L1” refers to a ratio (E1/E0) between 0.5-1.1, preferably between 0.7-1.0, and more preferably between 0.8-0.9, wherein E1 is the amount of PD-L1 expressed by the tumor and E0 is the amount of PD-L1 expressed by a normal tumor.

In another preferred embodiment, “low expression of PD-L1” refers to a ratio (E1/E0)≤1/2, preferably ≤1/3, and more preferably ≤1/4, wherein E1 is the amount of PD-L1 expressed by the tumor and E0 is the amount of PD-L1 expressed by a normal tumor.

In another preferred embodiment, the gene therapy vector exhibits a highly significant inhibitory effect on tumors with low expression of PD-L1 or no expression of PD-L1.

In another preferred embodiment, the expression of CMTM6 is downregulated or its activity is significantly reduced in the tumors or tumor cells after administration of the gene therapy vector.

In another preferred embodiment, the expression of PD-L1 is downregulated or its activity is significantly reduced in the tumors or tumor cells after administration of the gene therapy vector.

In another preferred embodiment, the tumors include but are not limited to: breast cancer, liver cancer, gastric cancer, large intestine cancer, melanoma, leukemia, lung cancer, renal tumors, small intestine cancer, prostate cancer, colorectal cancer, prostate cancer, cervical cancer, lymphoma, bone cancer, adrenal tumors, or bladder tumors.

In another preferred embodiment, the tumors are in situ tumors or metastatic tumors of the aforementioned tumors.

In another preferred embodiment, the tumor cells are located in vitro or in vivo.

In another preferred embodiment, the tumor cells include but are not limited to: breast cancer cells, liver cancer cells, colorectal cancer cells, melanoma cells, non-small cell lung cancer cells, etc.

In another preferred embodiment, the gene therapy vector comprises a viral vector and non-viral vector.

In another preferred embodiment, the viral vector includes but is not limited to: a lentivirus, an adenovirus, a retrovirus, an adeno-associated virus, etc.

In another preferred embodiment, the non-viral vector includes but is not limited to: a naked DNA, a liposome, a nanocarrier, etc.

In another preferred embodiment, the viral vector is a lentivirus.

In another preferred embodiment, the viral vector is an adeno-associated virus.

In another preferred embodiment, the gene therapy vector is selected from the group consisting of:
(Z1) a lentivirus for targeted inhibition of CMTM6 expression;
(Z2) an adeno-associated virus for targeted inhibition of CMTM6 expression;
(Z3) a lentivirus for simultaneous targeted inhibition of CMTM6 expression and PD-L1 expression;
(Z4) an adeno-associated virus for simultaneous targeted inhibition of CMTM6 expression and PD-L1 expression;
(Z5) any combinations of Z1-Z4 mentioned above.

In another preferred embodiment, the gene therapy vector carries or contains a coding sequence that targets DNA or RNA of CMTM6 and/or PD-L1.

In another preferred embodiment, the gene therapy vector selected from (Z1) or (Z2) carries or contains a coding sequence that targets DNA or RNA of CMTM6.

In another preferred embodiment, a coding sequence carried or contained in the gene therapy vector selected from (Z3) or (Z4) comprises (a) DNA or RNA targeting CMTM6; and (b) DNA or RNA targeting PD-L1.

In another preferred embodiment, a coding sequence carried or contained in the gene therapy vector is an oligonucleotide sequence capable of targeted degradation of CMTM6 gene expression in the cells.

In another preferred embodiment, the oligonucleotide sequence is a shRNA, siRNA, miRNA, sgRNA, or lncRNA, and most preferably a shRNA or sgRNA.

In another preferred embodiment, the oligonucleotide sequence is a shRNA or sgRNA.

In another preferred embodiment, the length of the shRNA is 17-62 nt, preferably 18-23 nt, and most preferably 19-21 nt.

In another preferred embodiment, the shRNA comprises a hairpin structure.

In another preferred embodiment, the length of the sgRNA is 18-23 nt, preferably 19-21 nt, and most preferably 20 nt.

In another preferred embodiment, the shRNA is selected from one or more (most preferably 1-3) of SEQ ID NOs: 1-12.

In another preferred embodiment, the shRNA comprises derived sequences obtained by substituting, adding, or deleting 1-3 nucleotides from any one of SEQ ID NOs: 1-12.

In another preferred embodiment, the sgRNA is selected from one or more (most preferably 1-3) of SEQ ID NOs: 13-15.

In another preferred embodiment, the sgRNA comprises derived sequences obtained by substituting, adding, or deleting 1-3 nucleotides from any one of SEQ ID NOs: 13-15.

In another preferred embodiment, a coding sequence carried or contained in the gene therapy vector is an oligonucleotide sequence capable of targeted degradation of PD-L1 gene expression in the cells.

In another preferred embodiment, the oligonucleotide sequence is a shRNA, siRNA, miRNA, sgRNA, or lncRNA, and most preferably a shRNA or sgRNA.

In another preferred embodiment, the oligonucleotide sequence is a shRNA.

In another preferred embodiment, the length of the shRNA is 17-62 nt, preferably 18-23 nt, and most preferably 19-21 nt.

In another preferred embodiment, the shRNA comprises a hairpin structure.

In another preferred embodiment, the shRNA is selected from one or more (most preferably 1-3) of SEQ ID NOs: 16-20.

In another preferred embodiment, the shRNA comprises derived sequences obtained by substituting, adding, or deleting 1-3 nucleotides from any one of SEQ ID NOs: 16-20.

In another preferred embodiment, a coding sequence carried or contained in the gene therapy vector comprises a dual-targeting nucleotide sequence that can simultaneously target CMTM6 and PD-L1, wherein the dual-targeting nucleotide sequence comprises:
(a) one or more selected from SEQ ID NOs: 1-15 or derived sequences thereof; and
(b) one or more selected from SEQ ID NOs: 16-20 or derived sequences thereof.

In another preferred embodiment, the derived sequences in (a) are derived sequences obtained by substituting, adding, or deleting 1-3 nucleotides from any one of SEQ ID NOs: 1-15.

In another preferred embodiment, the derived sequences in (b) are derived sequences obtained by substituting, adding, or deleting 1-3 nucleotides from any one of SEQ ID NOs: 16-20.

In another preferred embodiment, the sequences in group (a) and group (b) exhibit a synergistic inhibitory effect.

In another preferred embodiment, the dual-targeting nucleotide sequence exhibits a highly significant inhibitory effect on tumors with low expression of PD-L1 or no expression of PD-L1.

In the second aspect of the present invention, it provides a viral or non-viral vector capable of targeted inhibition of CMTM6 expression in tumors and/or cells, which carries or contains a coding sequence for inhibiting CMTM6 expression.

In another preferred embodiment, the viral or non-viral vector exhibits a highly significant inhibitory effect on tumors with low expression of PD-L1 or no expression of PD-L1.

In another preferred embodiment, “low expression of PD-L1” refers to a ratio (E1/E0)≤1/2, preferably ≤1/3, and more preferably ≤1/4, wherein E1 is the amount of PD-L1 expressed by the tumor and E0 is the amount of PD-L1 expressed by a normal tumor.

In another preferred embodiment, the viral vector comprises a lentivirus and adeno-associated virus.

In another preferred embodiment, the non-viral vector is selected from the group consisting of: a naked DNA, a liposome, a nanocarrier, etc.

In another preferred embodiment, the coding sequence for inhibiting CMTM6 expression targets DNA or RNA of CMTM6.

In another preferred embodiment, the coding sequence for inhibiting CMTM6 expression is an oligonucleotide sequence capable of targeted degradation of CMTM6 gene expression in the tumors and/or cells.

In another preferred embodiment, the oligonucleotide sequence is a shRNA, siRNA, miRNA, sgRNA, or lncRNA, and most preferably a shRNA or sgRNA.

In another preferred embodiment, the oligonucleotide sequence is a shRNA or sgRNA.

In another preferred embodiment, the coding sequence for inhibiting CMTM6 expression is a sgRNA or shRNA for targeted inhibition of CMTM6, comprising:
(i) one or more selected from SEQ ID NOs: 1-12 or derived sequences thereof; or
(ii) one or more selected from SEQ ID NOs: 13-15 or derived sequences thereof.

In another preferred embodiment, the derived sequences in (i) are derived sequences obtained by substituting, adding, or deleting 1-3 nucleotides from any one of SEQ ID NOs: 1-12.

In another preferred embodiment, the derived sequences in (ii) are derived sequences obtained by substituting, adding, or deleting 1-3 nucleotides from any one of SEQ ID NOs: 13-15.

In another preferred embodiment, the length of the shRNA is 17-62 nt, preferably 18-23 nt, and most preferably 19-21 nt.

In another preferred embodiment, the length of the sgRNA is 18-23 nt, preferably 19-21 nt, and most preferably 20 nt.

In another preferred embodiment, the shRNA is selected from one or more (most preferably 1-3) of SEQ ID NOs: 1-12.

In another preferred embodiment, the sgRNA is selected from one or more (most preferably 1-3) of SEQ ID NOs: 13-15.

In another preferred embodiment, the viral or non-viral vector can further inhibit the expression of PD-L1.

In another preferred embodiment, the viral or non-viral vector can further be used for combination therapy.

In another preferred embodiment, the scheme for combination therapy involves the use of the lentivirus in conjunction with drugs including but not limited to those selected from the group consisting of: immune checkpoint antibodies, immune agonists, chemotherapeutic drugs, lipid metabolism regulating drugs, glucose metabolism regulating drugs, and combinations thereof.

In another preferred embodiment, the species of the cells is human or mouse.

In another preferred embodiment, the cells are located in vitro or in vivo.

In another preferred embodiment, the cells are tumor cells or non-tumor cells.

In another preferred embodiment, the lentivirus exhibits inhibitory effects on tumors and/or tumor cells.

In another preferred embodiment, the inhibitory effects occur both in vivo and in vitro.

In another preferred embodiment, the tumor cells include but are not limited to: breast cancer cells, liver cancer cells, colorectal cancer cells, melanoma cells, non-small cell lung cancer cells, etc.

In another preferred embodiment, the expression of CMTM6 is downregulated or its activity is significantly reduced in the tumors or tumor cells after administration of the lentivirus.

In another preferred embodiment, the expression of PD-L1 is downregulated or its activity is significantly reduced in the tumors or tumor cells after administration of the lentivirus.

In the third aspect of the present invention, it provides a dual-targeting viral vector capable of simultaneous targeted inhibition of CMTM6 expression and PD-L1 expression in tumors and/or cells, wherein a coding sequence carried or contained in the dual-targeting viral vector is selected from the group consisting of:
(i) one or two selected from SEQ ID NOs: 1-15 or derived sequences thereof; and
(ii) one or two selected from SEQ ID NOs: 16-20 or derived sequences thereof.

In another preferred embodiment, the dual-targeting viral vector comprises a lentivirus and adeno-associated virus.

In another preferred embodiment, the derived sequences in (i) are derived sequences obtained by substituting, adding, or deleting 1-3 nucleotides from any one of SEQ ID NOs: 1-15.

In another preferred embodiment, the derived sequences in (ii) are derived sequences obtained by substituting, adding, or deleting 1-3 nucleotides from any one of SEQ ID NOs: 16-20.

In another preferred embodiment, the sequences in group (i) and group (ii) exhibit a synergistic inhibitory effect.

In another preferred embodiment, the dual-targeting viral vector exhibits a highly significant inhibitory effect on tumors with low expression of PD-L1 or no expression of PD-L1.

In another preferred embodiment, the dual-targeting viral vector can further be used for combination therapy.

In another preferred embodiment, the scheme for combination therapy involves the use of the dual-targeting viral vector in conjunction with drugs including but not limited to those selected from the group consisting of: immune checkpoint antibodies, immune agonists, chemotherapeutic drugs, lipid metabolism regulating drugs, glucose metabolism regulating drugs, and combinations thereof.

In another preferred embodiment, a coding sequence carried or contained in the dual-targeting viral vector comprises (a) DNA or RNA targeting CMTM6; and (b) DNA or RNA targeting PD-L1.

In another preferred embodiment, the dual-targeting viral vector exhibits inhibitory effects on tumors and/or tumor cells.

In another preferred embodiment, the inhibitory effects occur both in vivo and in vitro.

In the fourth aspect of the present invention, it provides a polynucleotide, which encodes the genome of a vector selected from the group consisting of: the vector of the second aspect of the present invention, or the dual-targeting viral vector of the third aspect of the present invention.

In another preferred embodiment, the polynucleotide comprises DNA, RNA, or cDNA.

In the fifth aspect of the present invention, it provides an expression vector, which comprises the polynucleotide of the fourth aspect of the present invention.

In another preferred embodiment, the expression vector comprises a plasmid vector, a viral vector, a liposome, a nanocarrier, or a combination thereof.

In another preferred embodiment, the viral vector comprises a baculovirus vector, a lentivirus expression vector, an adenovirus expression vector, a transposon expression vector, or a combination thereof.

In another preferred embodiment, the lentivirus expression vector is a lentiCRISPR lentivirus expression vector.

In another preferred embodiment, the lentivirus expression vector is a pLKO.1 lentivirus expression vector.

In another preferred embodiment, the adeno-associated virus vector is a pscAAV-EGFP-shRNA vector.

In another preferred embodiment, the adeno-associated virus vector is a pscAAV-EGFP-shRNA2 vector.

In another preferred embodiment, the expression vector further comprises an expression vector that has undergone modification(s).

In another preferred embodiment, the modification(s) include but are not limited to modification(s) to the viral capsid.

In another preferred embodiment, the modification is an RGD polypeptide modification.

In another preferred embodiment, the amino acid sequence of the RGD polypeptide is CDCRGDCFC.

In another preferred embodiment, the expression vector can further be used for combination therapy.

In another preferred embodiment, the scheme for combination therapy involves the use of the expression vector in conjunction with drugs including but not limited to those selected from the group consisting of: immune checkpoint antibodies, immune agonists, chemotherapeutic drugs, lipid metabolism regulating drugs, glucose metabolism regulating drugs, and combinations thereof.

In another preferred embodiment, the expression vector has a structure as shown in Formula I from 5′ to 3′:

Z0-Z1-Z2-Z3 (I)
wherein each “-” independently represents a bond or a nucleotide linkage sequence;
wherein Z0 is absent or an enhancer;
Z1 is a promoter element;
Z2 is a first nucleotide molecule that reduces the expression of a first target gene;
Z3 is an optional second nucleotide molecule that reduces the expression of a second target gene.

In another preferred embodiment, the expression vector comprises a promoter, an origin of replication, and a marker gene.

In another preferred embodiment, the promoter element comprises a constitutive promoter, an inducible promoter, or a specific promoter.

In another preferred embodiment, the promoter element is selected from the group consisting of: U6, CMV, EF1, and a combination thereof.

In another preferred embodiment, the first target gene and the second target gene are different.

In another preferred embodiment, the first target gene is CMTM6.

In another preferred embodiment, the second target gene is PD-L1.

In another preferred embodiment, the sequence of the first nucleotide molecule is selected from one or two of SEQ ID NOs: 1-15 or derived sequences thereof.

In another preferred embodiment, the derived sequences are derived sequences obtained by substituting, adding, or deleting 1-3 nucleotides from any one of SEQ ID NOs: 1-15.

In another preferred embodiment, the sequence of the second nucleotide molecule is selected from one or two of SEQ ID NOs: 16-20 or derived sequences thereof.

In another preferred embodiment, the derived sequences are derived sequences obtained by substituting, adding, or deleting 1-3 nucleotides from any one of SEQ ID NOs: 16-20.

In another preferred embodiment, the expression vector comprises a plasmid vector, a viral vector, a liposome, a nanocarrier, or a combination thereof.

In another preferred embodiment, the viral vector comprises a baculovirus vector, a lentivirus expression vector, an adenovirus expression vector, a transposon expression vector, or a combination thereof.

In another preferred embodiment, the expression vector is a lentivirus vector.

In another preferred embodiment, the expression vector is an adeno-associated virus vector.

In another preferred embodiment, the adeno-associated virus is a self-complementary adeno-associated virus.

In the sixth aspect of the present invention, it provides a host cell, which comprises the expression vector of the fifth aspect of the present invention, or has the polynucleotide of the fourth aspect of the present invention integrated into its genome.

In another preferred embodiment, the host cell comprises a prokaryotic cell or eukaryotic cell.

In another preferred embodiment, the host cell is selected from the group consisting of: Escherichia coli, yeast cells, HEK293T, HEK293F cells, CHO cells, etc.

In the seventh aspect of the present invention, it provides a method for producing the vector of the second aspect of the present invention or the dual-targeting viral vector of the third aspect of the present invention, comprising the steps of:
(a) under suitable conditions, introducing the polynucleotide of the fourth aspect of the present invention into a host cell or culturing the host cell of the sixth aspect of the present invention, thereby obtaining a culture of the vector or the dual-targeting viral vector;
(b) isolating and/or recovering the vector or the dual-targeting viral vector from the culture;
(c) optionally, purifying and/or modifying the vector or the dual-targeting viral vector obtained in step (b).

In the eighth aspect of the present invention, it provides a nucleic acid conjugate, which comprises:
(a) the polynucleotide of the fourth aspect of the present invention; and
(b) other coupled moiety.

In another preferred embodiment, the other coupled moiety is selected from the group consisting of: small molecule compounds, PEG, fluorescein, radioactive isotopes, fatty acid chains, protein fragments, polypeptides, and combinations thereof.

In another preferred embodiment, components (a) and (b) are operably linked.

In another preferred embodiment, the coupled moiety comprises chemical labels and biological labels.

In another preferred embodiment, the chemical labels are selected from isotopes, immunotoxins, and/or chemotherapeutic drugs.

In another preferred embodiment, the biological labels are selected from biotin, avidin, or enzyme labels.

In another preferred embodiment, the small molecule compounds are selected from drugs or toxins used in the treatment of tumors or autoimmune diseases.

In another preferred embodiment, the radioactive isotopes comprise:
(i) diagnostic isotopes selected from the group consisting of: Tc-99m, Ga-68, F-18, I-123, I-125, I-131, In-111, Ga-67, Cu-64, Zr-89, C-11, Lu-177, Re-188, and a combination thereof; and/or
(ii) therapeutic isotopes selected from the group consisting of: Lu-177, Y-90, Ac-225, As-211, Bi-212, Bi-213, Cs-137, Cr-51, Co-60, Dy-165, Er-169, Fm-255, Au-198, Ho-166, I-125, I-131, Ir-192, Fe-59, Pb-212, Mo-99, Pd-103, P-32, K-42, Re-186, Re-188, Sm-153, Ra223, Ru-106, Na24, Sr89, Tb-149, Th-227, Xe-133, Yb-169, Yb-177, and a combination thereof.

In another preferred embodiment, the radioactive isotopes include, but are not limited to, iodine-131, indium-111, and lutetium-177.

In another preferred embodiment, the protein fragments include, but are not limited to, antibody Fc, biotin, avidin, HRP, antibodies, enzymes, cytokines, and other bioactive proteins or polypeptides.

In another preferred embodiment, the coupled moiety is a detectable marker.

In another preferred embodiment, the coupled moiety is selected from the group consisting of: fluorescent or luminescent markers, radioactive markers, MRI (Magnetic Resonance Imaging) or CT (Electronic computer X-ray tomography technology) contrast agents, enzymes capable of generating detectable products, radionuclides, biotoxins, cytokines (such as IL-2, etc.), antibodies, antibody Fc fragments, antibody scFv fragments, gold nanoparticles/nanorods, viral particles, liposomes, magnetic nanoparticles, prodrug-activating enzymes (e.g., DT-diaphorase (DTD) or biphenyl hydrolase-like protein (BPHL)), or any form of nanoparticles.

In another preferred embodiment, the polypeptide molecules or fragments include, but are not limited to: polypeptide molecules or fragments targeting PD-1, IL-4R, IL-4Rα, TNF-α, VEGF, 4-1BB, CD47, TIM3, CTLA4, IL-17A, CD19, CD22, CD28, CD38, CD40, CD47, B7-H3, TSLP, BCMA, GLP-1, Trop2, TIGIT, LAG-3, FGL1, HER2.

In another preferred embodiment, the polypeptide molecule is an RGD polypeptide or its derivative.

In another preferred embodiment, the polypeptide molecules or fragments with therapeutic functions comprise single-chain antibodies (scFv), double-chain antibodies, monoclonal antibodies, or chimeric antibodies.

In another preferred embodiment, the fusion protein further comprises a tag sequence for assisting in expression and/or purification.

In another preferred embodiment, the tag sequence is selected from the group consisting of: 6His tag, GGGS sequence, and FLAG tag.

In another preferred embodiment, the fusion protein comprises bispecific antibodies or chimeric antibodies.

In the ninth aspect of the present invention, it provides a pharmaceutical formulation, which comprises:
(a) the expression vector of the fifth aspect of the present invention or the nucleic acid conjugate of the eighth aspect of the present invention; and
(b) a pharmaceutically acceptable carrier.

In another preferred embodiment, the formulation is in a liquid dosage form.

In another preferred embodiment, the formulation is an injection.

In another preferred embodiment, the expression vector comprises a lentivirus vector, an adeno-associated virus vector, or a combination thereof.

In the tenth aspect of the present invention, it provides a pharmaceutical composition, which comprises:
(a) the pharmaceutical formulation of the ninth aspect of the present invention; and
(b) other bioactive drugs, such as drugs for treating tumors.

In another preferred embodiment, the pharmaceutical composition comprises single drugs, compound drugs, or synergistic drugs.

In another preferred embodiment, the other bioactive drugs comprise immune checkpoint antibodies or gene therapy vectors, immune agonist drugs, chemotherapeutic drugs, lipid metabolism regulating drugs, or glucose metabolism regulating drugs.

In another preferred embodiment, the gene therapy vector refers to a gene therapy vector targeting immune checkpoints.

In another preferred embodiment, the immune checkpoints are selected from the group consisting of: PD-1, PD-L1, CTLA-4, B7-H3, LAG-3, VISTA, CD47, TIM-3, TIGIT, BTLA, Siglec-15, etc.

In another preferred embodiment, the targets of the immune checkpoint antibodies include, but are not limited to: PD-1, PD-L1, CTLA-4, B7-H3, LAG-3, VISTA, CD47, TIM-3, TIGIT, BTLA, Siglec-15, etc.

In another preferred embodiment, the immune agonist drugs are TLR receptor agonists, CD40 agonistic antibodies, STING agonists, CD3 antibodies, CD28 antibodies, etc.

In another preferred embodiment, the TLR receptor agonist is a TLR7 agonist.

In another preferred embodiment, the chemotherapeutic drugs are selected from doxorubicin, paclitaxel, cisplatin, carboplatin, gemcitabine, pemetrexed, methotrexate, oxaliplatin, fluorouracil, etc.

In another preferred embodiment, the lipid metabolism regulating drugs are selected from statins or ezetimibe, etc.

In another preferred embodiment, the statin is fluvastatin.

In another preferred embodiment, the glucose metabolism regulating drug is metformin.

In another preferred embodiment, the pharmaceutical composition is used for anti-tumor treatment.

In another preferred embodiment, the tumors are selected from, but not limited to: breast cancer, liver cancer, gastric cancer, large intestine cancer, leukemia, lung cancer, renal tumors, small intestine cancer, prostate cancer, colorectal cancer, prostate cancer, cervical cancer, lymphoma, bone cancer, adrenal tumors, or bladder tumors.

In another preferred embodiment, the tumors are in situ tumors or metastatic tumors of the aforementioned tumors.

In another preferred embodiment, the tumors are resistant to immune checkpoint antibody therapy.

In another preferred embodiment, the targets of the immune checkpoint antibodies include, but are not limited to: PD-1, PD-L1, CTLA-4, B7-H3, LAG-3, VISTA, CD47, TIM-3, TIGIT, BTLA, Siglec-15, etc.

In another preferred embodiment, the tumors are tumors with expression of PD-L1 and tumors with no expression of PD-L1.

In another preferred embodiment, the tumors are selected from the group consisting of: tumors with high expression of PD-L1, tumors with medium expression of PD-L1, and tumors with low expression of PD-L1.

In another preferred embodiment, the tumors are tumors with medium expression of PD-L1 or tumors with low expression of PD-L1.

In another preferred embodiment, the tumors are tumors with low expression of PD-L1.

In another preferred embodiment, “high expression of PD-L1” refers to a ratio (E1/E0)>1, preferably ≥1.5, and more preferably ≥2.0, wherein E1 is the amount of PD-L1 expressed by the tumor and E0 is the amount of PD-L1 expressed by a normal tumor.

In another preferred embodiment, “medium expression of PD-L1” refers to a ratio (E1/E0) between 0.5-1.1, preferably between 0.7-1.0, and more preferably between 0.8-0.9, wherein E1 is the amount of PD-L1 expressed by the tumor and E0 is the amount of PD-L1 expressed by a normal tumor.

In another preferred embodiment, “low expression of PD-L1” refers to a ratio (E1/E0)≤1/2, preferably ≤1/3, and more preferably ≤1/4, wherein E1 is the amount of PD-L1 expressed by the tumor and E0 is the amount of PD-L1 expressed by a normal tumor.

In another preferred embodiment, the pharmaceutical formulation and pharmaceutical composition are administered in a manner selected from the group consisting of: subcutaneous injection, intradermal injection, intramuscular injection, intravenous injection, intraperitoneal injection, microneedle injection, oral administration, oral and nasal spray, and nebulized inhalation.

In another preferred embodiment, the pharmaceutical formulation and pharmaceutical composition are administered in a dosage form selected from the group consisting of: liquid, solid, or gel.

In the eleventh aspect of the present invention, it provides a method for preventing and/or treating tumors, comprising the step of: administering the vector of the second aspect of the present invention, the dual-targeting viral vector of the third aspect of the present invention, the expression vector of the fifth aspect of the present invention, the nucleic acid conjugate of the eighth aspect of the present invention, the pharmaceutical formulation of the ninth aspect of the present invention, or the pharmaceutical composition of the tenth aspect of the present invention, to a subject in need thereof.

In another preferred embodiment, the subject is a human or a non-human mammal.

In another preferred embodiment, the non-human mammal is a rodent, such as a mouse.

In another preferred embodiment, the expression vectors are viral vectors and non-viral vectors.

In another preferred embodiment, the viral vector includes but is not limited to: a lentivirus, an adenovirus, a retrovirus, an adeno-associated virus, etc.

In another preferred embodiment, the non-viral vector includes but is not limited to: a naked DNA, a liposome, a nanocarrier, etc.

In another preferred embodiment, the administration comprises injecting a lentivirus vector, an adeno-associated virus vector, or a combination thereof.

In another preferred embodiment, the tumors are resistant to immune checkpoint antibody therapy.

In another preferred embodiment, the targets of the immune checkpoint antibodies include, but are not limited to: PD-1, PD-L1, CTLA-4, B7-H3, LAG-3, VISTA, CD47, TIM-3, TIGIT, BTLA, Siglec-15, etc.

In another preferred embodiment, the immune checkpoint antibodies are PD-L1 antibodies and CTLA-4 antibodies.

In another preferred embodiment, the tumors are tumors with expression of PD-L1 and tumors with no expression of PD-L1.

In another preferred embodiment, the tumors are selected from the group consisting of: tumors with high expression of PD-L1, tumors with medium expression of PD-L1, and tumors with low expression of PD-L1.

In another preferred embodiment, the tumors are tumors with medium expression of PD-L1 or tumors with low expression of PD-L1.

In another preferred embodiment, the tumors are tumors with low expression of PD-L1.

In another preferred embodiment, “high expression of PD-L1” refers to a ratio (E1/E0)>1, preferably ≥1.5, and more preferably ≥2.0, wherein E1 is the amount of PD-L1 expressed by the tumor and E0 is the amount of PD-L1 expressed by a normal tumor.

In another preferred embodiment, “medium expression of PD-L1” refers to a ratio (E1/E0) between 0.5-1.1, preferably between 0.7-1.0, and more preferably between 0.8-0.9, wherein E1 is the amount of PD-L1 expressed by the tumor and E0 is the amount of PD-L1 expressed by a normal tumor.

In another preferred embodiment, “low expression of PD-L1” refers to a ratio (E1/E0)≤1/2, preferably ≤1/3, and more preferably ≤1/4, wherein E1 is the amount of PD-L1 expressed by the tumor and E0 is the amount of PD-L1 expressed by a normal tumor.

In another preferred embodiment, the tumors include but are not limited to: breast cancer, liver cancer, gastric cancer, large intestine cancer, melanoma, leukemia, lung cancer, renal tumors, small intestine cancer, prostate cancer, colorectal cancer, prostate cancer, cervical cancer, lymphoma, bone cancer, adrenal tumors, or bladder tumors.

In another preferred embodiment, the tumors are in situ tumors or metastatic tumors of the aforementioned tumors.

In another preferred embodiment, the mode of administration is selected from the group consisting of: subcutaneous injection, intradermal injection, intramuscular injection, intravenous injection, intraperitoneal injection, microneedle injection, oral administration, oral and nasal spray, and nebulized inhalation.

In another preferred embodiment, the dosage form for administration is selected from the group consisting of: liquid, solid, or gel.

It should be understood that within the scope of the present invention, the above-mentioned technical features of the present invention and the technical features specifically described in the following (such as the examples) can be combined with each other to form a new or preferred technical solution, which is not redundantly repeated one by one herein due to space limitation.

### DESCRIPTION OF THE DRAWINGS

FIG. 1 shows the coding pattern diagram of the lentivirus for knocking out CMTM6 expression of the present invention.

FIG. 2 shows Western blot exposure images demonstrating that the sgRNA for knocking out CMTM6 expression of the present invention can effectively delete the CMTM6 expression in tumor cells in vitro.

FIG. 3 shows the tumor growth curves and endpoint tumor weights in mice, demonstrating that the lentivirus for knocking out CMTM6 expression of the present invention can significantly inhibit the tumor growth of CT26 colorectal cancer in vivo.

FIG. 4 shows the tumor growth curves and endpoint tumor weights in mice, demonstrating that the lentivirus for knocking out CMTM6 expression of the present invention can significantly inhibit the tumor growth of MC38 colorectal cancer in vivo.

FIG. 5 shows the tumor growth curves and endpoint tumor weights in mice, demonstrating that the lentivirus for knocking out CMTM6 expression of the present invention can significantly inhibit the growth of B16F10 melanoma in vivo.

FIG. 6 shows the tumor growth curves and endpoint tumor weights in mice, demonstrating that the lentivirus for knocking out CMTM6 expression of the present invention can significantly inhibit the tumor growth of Hepa1-6 liver cancer in vivo.

FIG. 7 shows the tumor growth curves and endpoint tumor weights in mice, demonstrating that the lentivirus for knocking out CMTM6 expression of the present invention can significantly inhibit the tumor growth of LLC non-small cell lung cancer in vivo.

FIG. 8 shows the coding pattern diagram of the lentivirus for knocking out CMTM6 expression and PD-L1 expression of the present invention.

FIG. 9 shows the tumor growth curves in mice, demonstrating that the lentivirus for knocking out CMTM6 expression of the present invention can significantly inhibit the tumor growth of PD-L1-deficient CT26 colorectal cancer in vivo.

FIG. 10 shows the tumor growth curves and endpoint tumor weights in mice, demonstrating that the lentivirus for knocking out CMTM6 expression of the present invention can elicit anti-tumor immune memory in vivo.

FIG. 11 shows the tumor growth curves and endpoint tumor weights in mice, demonstrating that the lentivirus for knocking out CMTM6 expression of the present invention can significantly inhibit the growth of PD-L1-deficient B16F10 melanoma in vivo.

FIG. 12 shows the tumor growth curves and endpoint tumor weights in mice, demonstrating that the lentivirus for knocking out CMTM6 expression of the present invention can significantly inhibit the growth of PD-1 and PD-L1-deficient non-responsive MC38 colorectal cancer in vivo.

FIG. 13 shows the tumor growth curves and endpoint tumor weights in mice, demonstrating that the lentivirus for knocking out CMTM6 expression of the present invention can significantly enhance the efficacy of PD-L1 antibody in 4T1 breast cancer resistant to immune checkpoint therapy in vivo.

FIG. 14 shows the tumor growth curves and endpoint tumor weights in mice, demonstrating that the lentivirus for knocking out CMTM6 expression of the present invention can significantly enhance the efficacy of PD-L1 and CTLA-4 antibodies in LLC non-small cell lung cancer resistant to immune checkpoint therapy in vivo.

FIG. 15 shows the photographs of mouse lungs and lung coefficients, demonstrating that the lentivirus for knocking out CMTM6 expression of the present invention can significantly inhibit the lung metastasis of B16F10 melanoma in vivo.

FIG. 16 shows the photographs of mouse lungs and lung coefficients, demonstrating that the lentivirus for knocking out CMTM6 expression of the present invention can significantly inhibit the lung metastasis of 4T1 breast cancer in vivo.

FIG. 17 shows the coding pattern diagram of the lentivirus for interfering with CMTM6 expression of the present invention.

FIG. 18 shows the tumor growth curves and endpoint tumor weights in mice, demonstrating that the lentivirus for interfering with CMTM6 expression and the lentivirus for interfering with PD-L1 expression of the present invention can significantly inhibit the tumor growth of CT26 colorectal cancer in vivo.

FIG. 19 shows the coding pattern diagram of the adeno-associated virus for interfering with CMTM6 expression of the present invention.

FIG. 20 shows the coding pattern diagram of the adeno-associated virus for knocking out CMTM6 expression of the present invention.

FIG. 21 shows flow cytometry data demonstrating that the adeno-associated virus of the present invention can infect multiple types of tumor cells in vitro.

FIG. 22 shows fluorescence images demonstrating that the adeno-associated virus of the present invention can infect multiple types of tumor cells in vitro.

FIG. 23 shows Western blot relative quantification results demonstrating that the adeno-associated virus of the present invention can reduce CMTM6 expression in tumor cells in vitro.

FIG. 24 shows flow cytometry data demonstrating that the adeno-associated virus of the present invention can infect tumor cells in vivo.

FIG. 25 shows the tumor growth curves and endpoint tumor weights in mice, demonstrating that the adeno-associated virus for interfering with CMTM6 expression of the present invention can inhibit the tumor growth of CT26 colorectal cancer in vivo.

FIG. 26 shows the tumor growth curves and endpoint tumor weights in mice, demonstrating that the adeno-associated virus for interfering with CMTM6 expression of the present invention can inhibit the tumor growth of B16 melanoma in vivo.

FIG. 27 shows the tumor growth curves and endpoint tumor weights in mice, demonstrating that the adeno-associated virus for interfering with CMTM6 expression of the present invention can inhibit the tumor growth of Hepa1-6 liver cancer in vivo.

FIG. 28 shows the coding pattern diagram of the adeno-associated virus for simultaneously interfering with CMTM6 and PD-L1 expressions of the present invention.

FIG. 29 shows flow cytometry analysis demonstrating that the shRNA for interfering with PD-L1 expression can reduce PD-L1 levels in CT26 tumor cells.

FIG. 30 shows the tumor growth curves and endpoint tumor weights in mice, demonstrating that the adeno-associated virus for interfering with CMTM6 expression, the adeno-associated virus for interfering with PD-L1 expression, and the adeno-associated virus for simultaneously interfering with CMTM6 and PD-L1 expressions of the present invention can inhibit the tumor growth of CT26 colorectal cancer in vivo.

FIG. 31 shows photographs of dissected tumors, demonstrating that the adeno-associated virus for interfering with CMTM6 expression, the adeno-associated virus for interfering with PD-L1 expression, and the adeno-associated virus for simultaneously interfering with CMTM6 and PD-L1 expressions of the present invention can inhibit the tumor growth of CT26 colorectal cancer in vivo.

FIG. 32 shows the tumor growth curves in mice, demonstrating that the adeno-associated virus for interfering with CMTM6 expression of the present invention combined with the PD-L1 antibody drug can inhibit the tumor growth of CT26 colorectal cancer in vivo.

FIG. 33 shows the tumor growth curves in mice, demonstrating that the adeno-associated virus for interfering with CMTM6 expression of the present invention combined with the chemotherapy drug can inhibit the tumor growth of CT26 colorectal cancer in vivo.

FIG. 34 shows the tumor growth curves in mice, demonstrating that the adeno-associated virus for interfering with CMTM6 expression of the present invention combined with the immune agonist drug can inhibit the tumor growth of CT26 colorectal cancer in vivo.

FIG. 35 shows the tumor growth curves in mice, demonstrating that the adeno-associated virus for interfering with CMTM6 expression of the present invention combined with the glucose metabolism regulating drug can inhibit the tumor growth of CT26 colorectal cancer in vivo.

FIG. 36 shows the tumor growth curves in mice, demonstrating that the adeno-associated virus for interfering with CMTM6 expression of the present invention combined with the lipid metabolism regulating drug can inhibit the tumor growth of CT26 colorectal cancer in vivo.

FIG. 37 shows that the adeno-associated virus for interfering with CMTM6 expression of the present invention can improve the tumor immune microenvironment.

FIG. 38 shows the coding pattern diagram of the RGD polypeptide-modified adeno-associated virus for interfering with CMTM6 expression of the present invention.

FIG. 39 shows the endpoint tumor weights and tumor growth curves of mouse dissected tumors, demonstrating that the RGD polypeptide-modified adeno-associated virus for interfering with CMTM6 expression of the present invention can inhibit the tumor growth of CT26 colorectal cancer through in vivo systemic administration.

FIG. 40 shows the growth curves of human-derived RKO tumors, demonstrating that the lentivirus for knocking out CMTM6 expression of the present invention can significantly inhibit the tumor growth of RKO colon cancer in vivo.

### DETAILED DESCRIPTION

After extensive and in-depth research and a large number of screenings, the inventors have developed a gene therapy vector (e.g., including lentivirus and adeno-associated virus) that interferes with the expression of CMTM6 for the first time. The lentivirus and adeno-associated virus constructed by the present invention can be used as gene vectors for sgRNA or shRNA that can degrade the expression of CMTM6 gene, which can reduce the expression of CMTM6 in vitro and in vivo. In addition, the inventors further selected colorectal cancer, melanoma, liver cancer, breast cancer, non-small cell lung cancer and other tumor models and metastatic models (including mouse tumors and human-derived tumors) to evaluate the anti-tumor effects of the developed lentiviruses and adeno-associated viruses that interfere with CMTM6 expression. At the same time, modifications and scheme tests for combination therapy were also conducted on lentiviruses and adeno-associated viruses, and the therapeutic effects and immune microenvironment regulation of the developed lentiviruses and adeno-associated viruses that interfere with or downregulate CMTM6 expression were evaluated for tumors with low or no PD-L1 expression and tumors resistant to immune checkpoint therapy. On this basis, the present invention is completed.

Experiments show that the gene therapy vector of the present invention has a significant inhibitory effect on the growth and metastasis of a variety of tumors, and the combination therapy also shows excellent anti-tumor effects, demonstrating broad clinical application prospects.

In addition, the gene therapy vector of the present invention has an excellent therapeutic effect (including inhibition of tumor growth and/or metastasis) for tumors that are not suitable for treatment with conventional immune checkpoint drugs such as PD-L1 antibodies, and can be applied to some drug-resistant tumors, especially refractory tumors that have shown ineffectiveness for treatment with PD-L1 antibodies.

### Terms

To facilitate a better understanding of the present disclosure, certain terms are first defined. As used in the present application, each of the following terms shall have the meaning given below, unless otherwise expressly defined herein. Other definitions were elaborated throughout the application.

As used herein, the terms “contain” or “comprise (include)” can be open-ended, semi-enclosed, and enclosed. In other words, the terms also comprise “substantially composed of . . . ”, or “composed of . . . ”.

Sequence identity is determined by comparing two aligned sequences along a predetermined comparison window (which can be 50%, 60%, 70%, 80%, 90%, 95%, or 100% of the length of a reference nucleotide sequence or protein), and determining the number of positions where identical residues appear. Usually, this is expressed as a percentage. The measurement of sequence identity of nucleotide sequences is a well-known method for those skilled in the art.

As used herein, the terms “subject”, “subject in need” refer to any mammal or non-mammal. Mammals include, but are not limited to, humans, vertebrates such as rodents, non-human primates, cattle, horses, dogs, cats, pigs, sheep, and goats.

As used herein, the terms “gene therapy vector”, “gene therapy vector of the present invention”, “gene therapy vector interfering with CMTM6 expression”, “gene vector”, etc. are used interchangeably, and all refer to gene therapy vectors constructed in the present application that can reduce the expression of CMTM6 in vitro and in vivo.

As known to those skilled in the art, nucleic acid conjugates and fusion expression products include: conjugates formed by combining drugs, toxins, cytokines, radionuclides, enzymes and other diagnostic or therapeutic molecules with the nucleic acid molecules or fragments of the present invention. The present invention further comprises cell surface markers or antigens bound to the nucleic acid molecules or fragments.

As used herein, the terms “fragment”, “derivative” and “analogue” refer to polypeptides that maintain essentially the same biological function or activity as the nucleotide molecules of the present invention. The polypeptide fragment, derivative or analogue of the present invention may be (i) a polypeptide in which one or more conserved or non-conserved amino acid residues (preferably conserved amino acid residues) are substituted, and such substituted amino acid residues may or may not be encoded by the genetic code, or (ii) a polypeptide with substituent groups in one or more amino acid residues, or (iii) a polypeptide formed by the fusion of a mature polypeptide with another compound (e.g., a compound that extends the half-life of the polypeptide, e.g., polyethylene glycol), or (iv) a polypeptide formed by the fusion of an additional amino acid sequence to this polypeptide sequence (e.g., a leader sequence or secretion sequence or a sequence or protein precursor sequence used to purify this polypeptide, or a fusion protein formed with a 6His tag). According to the teachings herein, these fragments, derivatives and analogues fall within the realm of knowledge of those skilled in the art.

The nucleotide molecule of the present invention can be in DNA form or RNA form. DNA forms comprise cDNA, genomic DNA, or synthetic DNA. DNA can be single-stranded or double-stranded. DNA can be either a coding strand or a non-coding strand.

Once the relevant sequences have been obtained, the relevant sequences can be obtained in large quantities using the recombination method. This typically involves cloning them into vectors, transferring them into cells, and then isolating the relevant sequences from proliferating host cells by conventional methods. The biomolecules (nucleic acids, proteins, etc.) involved in the present invention include biomolecules in isolated form.

At present, the sequence of the nucleotide molecule (or its fragment, or its derivative) of the present invention can be obtained completely by chemical synthesis. This sequence can then be introduced into a variety of existing nucleic acid molecules (or e.g., vectors) and cells known in the art. In addition, mutations can be introduced into the nucleotide sequence of the present invention by chemical synthesis.

The invention also relates to a vector containing the appropriate sequence described above and an appropriate promoter or control sequence. These vectors can be used to transform appropriate host cells to enable them to express proteins.

Host cells can be prokaryotic cells, such as bacterial cells; or lower eukaryotic cells, such as yeast cells; or higher eukaryotic cells, such as mammalian cells. Representative examples are: Escherichia coli, Streptomyces; bacterial cells of Salmonella typhimurium; fungal cells such as yeast; insect cells of Drosophila S2 or Sf9; animal cells such as CHO, COS7, 293 cells, etc.

Transformation of host cells with recombinant nucleic acids can be performed using conventional techniques that are well known to those skilled in the art. When the host is a prokaryote such as Escherichia coli, competent cells that can absorb nucleic acid molecules can be harvested after the exponential growth phase and then treated with the CaCl₂) method, the steps used are well known in the art. Another method is to use MgCl2. If desired, the transformation can also be carried out by electroporation. When the host is a eukaryote, the following DNA transfection methods can be used: calcium phosphate co-precipitation, conventional mechanical methods such as microinjection, electroporation, liposome packaging, etc.

The obtained transformant can be cultured by conventional methods to express the polypeptide encoded by the gene of the present invention. Depending on the host cells used, the media used in the culture can be selected from a variety of conventional media. Perform the culture under conditions suitable for host cell growth. After the host cells have grown to the appropriate cell density, the selected promoter is induced using appropriate methods (such as temperature conversion or chemical induction), and the cells are cultured for another period of time.

The recombinant polypeptides in the above methods can be expressed intracellularly, on the cell membrane, or secreted extracellularly. If desired, recombinant proteins can be isolated and purified by a variety of isolation methods using their physical, chemical, and other properties. These methods are well known to those skilled in the art. Examples of these methods include, but are not limited to: conventional renaturation, treatment with protein precipitants (salting-out method), centrifugation, osmotic disruption of bacteria, ultra-processing, ultracentrifugation, molecular sieve chromatography (gel filtration), adsorption chromatography, ion exchange chromatography, high-performance liquid chromatography (HPLC), and various other liquid chromatography techniques, as well as combinations of these methods.

The nucleic acid or vector of the present invention may be used alone or in combination or conjugated with a detectable marker (for diagnostic purposes), a therapeutic agent, a PK (protein kinase) modified moiety, or a combination of any of the above.

Detectable markers for diagnostic purposes include, but are not limited to: fluorescent or luminescent markers, radioactive markers, MRI (Magnetic Resonance Imaging) or CT (Electronic computer X-ray tomography technology) contrast agents, or enzymes capable of generating detectable products.

Therapeutic agents that can be bound or conjugated with nucleic acids or vectors of the present invention include but are not limited to: 1. radionuclides; 2. biotoxins; 3. cytokines such as IL-2, etc.; 4. gold nanoparticles/nanorods; 5. viral particles; 6. liposomes; 7. magnetic nanoparticles; 8. prodrug-activating enzymes (e.g., DT-diaphorase (DTD) or biphenyl hydrolase-like protein (BPHL)), etc.

### Gene Therapy and Vectors Thereof

Gene therapy is a means of introducing a target gene into a target cell to achieve the therapeutic goal. Existing gene therapy vectors include adenovirus vectors, retrovirus vectors, lentivirus vectors, and adeno-associated virus vectors. Adeno-associated virus (AAV) is a simple, non-envelope, DNA-deficient type of virus. The life cycle of AAV is dependent on replicating viruses such as adenovirus and herpes simplex virus. AAV has good tissue specificity, efficient diffusion, low immunogenicity, high safety and stability. Therefore, recombinant adeno-associated virus is one of the promising viral vectors. Lentivirus vectors are modified from type I human acquired immunodeficiency virus (HIV-I), which can infect dividing cells and non-dividing cells. The encoded genes can be integrated into the genome of target cells and the coding capacity is large, making them also one of the promising viral vectors.

### CMTM6

As used herein, the terms “CMTM6 protein”, “polypeptide”, “protein of the present invention”, and “human CMTM6 protein” have the same meaning and are used interchangeably herein.

CMTM6 is a member of the human chemokine-like factor superfamily (CMTM family). CMTM6 contains a four-transmembrane MARVEL domain. On the surface of tumor cells, CMTM6 can act as a post-translational modification regulatory molecule for of PD-L1, interact with PD-L1 and maintain the expression level of PD-L1 on the cell membrane by inhibiting its ubiquitin-proteasome degradation pathway and endosome-lysosome degradation pathway.

The inventors unexpectedly discovered that the growth of tumor cells can be effectively inhibited by downregulating the expression of the protein of the present invention. Especially for tumor cells with low expression or no expression of PD-L1, the present invention can more effectively inhibit the tumors by inhibiting the expression of CMTM6.

### The First Nucleotide Molecule

In the present invention, a first nucleotide molecule refers to a nucleotide molecule capable of reducing the expression of a first target gene (i.e., CMTM6).

In a preferred embodiment, the sequence of the first nucleotide molecule is shown in SEQ ID NOs: 1-15.

### The Second Nucleotide Molecule

In the present invention, a second nucleotide molecule refers to a nucleotide molecule capable of reducing the expression of a second target gene (i.e., PD-L1).

In a preferred embodiment, the sequence of the second nucleotide molecule is shown in SEQ ID NOs: 16-20.

### Expression Vectors

The present invention also provides an expression vector, which comprises the polynucleotide of the present invention. The expression vector usually further contains a promoter, an origin of replication, and/or a marker gene, etc. The methods well-known to those skilled in the art can be used to construct the expression vector required for the present invention. These methods include in vitro recombinant DNA technology, DNA synthesis technology, in vivo recombinant technology, etc. The expression vector preferably contains one or more selective marker genes to provide phenotypic traits for selecting transformed host cells, such as kanamycin, gentamicin, hygromycin, and ampicillin resistance.

In the present invention, representative promoters include (but are not limited to): U6, CMV, EF1 promoters or combinations thereof.

### Treatment Method

The present invention also provides a method for treating tumors, namely, administering a safe and effective amount of the expression vector of the seventh aspect of the present invention, the nucleic acid conjugate of the tenth aspect of the present invention, the pharmaceutical formulation of the eleventh aspect of the present invention, or the pharmaceutical composition of the twelfth aspect of the present invention to a subject in need thereof, thereby treating the tumors.

The main advantages of the present invention include:
(a) The present invention provides for the first time a gene therapy vector that can interfere with the expression of CMTM6 and its anti-tumor application. The gene therapy vectors that interfere with CMTM6 expression, such as lentiviruses and adeno-associated viruses of the present invention, have significant efficacy against the growth and metastasis of tumors such as colorectal cancer, melanoma, liver cancer, breast cancer, and non-small cell lung cancer (including mouse tumors and human-derived tumors).
(b) The gene therapy vectors that interfere with CMTM6 expression, such as lentiviruses and adeno-associated viruses, provided by the present invention have significant efficacy against tumors resistant to immune checkpoint (such as PD-1/PD-L1/CTLA-4) antibody therapy, and also have significant efficacy against tumors with low or no expression of PD-L1, showing the prospect of clinical application.
(c) The present invention provides for the first time the anti-tumor application of a combination therapy regimen utilizing a gene therapy vector that can interfere with CMTM6 expression. Test results have shown that the combination therapy of the gene therapy vector with immune checkpoint antibodies/immune agonists/chemotherapeutic drugs/lipid metabolism regulating drugs/glucose metabolism regulating drugs exhibits excellent anti-tumor effects.
(d) The present invention provides for the first time a gene therapy vector that can simultaneously interfere with the expression of CMTM6 and PD-L1, demonstrating an excellent combined anti-tumor effect that can regress the in vivo growth of tumors on the one hand and stimulate anti-tumor immune memory on the other hand, indicating broad clinical application prospects.
(e) The invention provides for the first time a modified derivative of a gene therapy vector that interferes with the expression of CMTM6, and successfully realizes a more convenient systemic administration.
(f) The gene therapy vector that interferes with the expression of CMTM6 provided by the present invention for the first time can remodel the tumor immune microenvironment, and stimulate the anti-tumor immune response of CD8⁺ T and NK cells, etc., revealing the value of CMTM6 as a tumor immunomodulatory target.
(g) Compared with current tumor immunotherapy represented by immune checkpoint blockade therapy, the gene therapy vector of the present invention has greatly improved the efficacy, and also improved the response rate, providing a new technical means for the existing tumor treatment methods.

The present invention will be further illustrated below with reference to the specific examples. It should be understood that these examples are only to illustrate the present invention, not to limit the scope of the present invention. The conditions of the experimental methods not specifically indicated in the following examples are usually in accordance with conventional conditions as described in e.g., Sambrook et al., Molecular Cloning: A Laboratory Manual (New York: Cold Spring Harbor Laboratory Press, 1989), or according to the conditions recommended by the manufacturers. Percentages and parts are by weight unless otherwise stated.

Nucleotide sequence

>SEQ ID NO: 1 mCMTM6_shRNA_1
TGCCTAACAGAAAGCGTGT
>SEQ ID NO: 2 mCMTM6_shRNA_2
CCGGAGATTTAATGAGTGTTT
>SEQ ID NO: 3 mCMTM6_shRNA_3
CCTCAGCTGAAATTGCTGCAA
>SEQ ID NO: 4 hCMTM6_shRNA_1
CCGGCCTTTCTTCTGAGT
>SEQ ID NO: 5 hCMTM6_shRNA_2
CCGGCTTTCTTCTGAGT
>SEQ ID NO: 6 hCMTM6_shRNA_3
CCTTTCTTCTGAGTCTCCTTA
>SEQ ID NO: 7 mCMTM6_shRNA_4
TGCCTAACAGAAAGCGTGTCTCGAGACACGCTTTCTGTTAGGCATTTT
TG
>SEQ ID NO: 8 mCMTM6_shRNA_5
CCGGAGATTTAATGAGTGTTTCTCGAGAAACACTCATTAAATCTCCGG
TTTTTG
>SEQ ID NO: 9 mCMTM6_shRNA_6
CCTCAGCTGAAATTGCTGCAACTCGAGTTGCAGCAATTTCAGCTGAGG
TTTTTG
>SEQ ID NO: 10 hCMTM6_shRNA_4
CCGGCCTTTCTTCTGAGTCTCCTTACTCGAGTAAGGAGACTCAGAAGA
AAGGTTTTTG
>SEQ ID NO: 11 hCMTM6_shRNA_5
CCGGCTTTCTTCTGAGTCTCCTTATCTCGAGATAAGGAGACTCAGAAG
AAAGTTTTTTG
>SEQ ID NO: 12 hCMTM6_shRNA_6
CCTTTCTTCTGAGTCTCCTTACTCGAGTAAGGAGACTCAGAAGAAAGG
TTTTTTG
>SEQ ID NO: 13 mCMTM6_sgRNA
CCTGGCCGCCTACTTCGTCC
>SEQ ID NO: 14 hCMTM6_sgRNA_1
CCGGGTCCTCCTCCGTAGTG
>SEQ ID NO: 15 hCMTM6_sgRNA_2
TCACAATGTACTTTATGTGG
>SEQ ID NO: 16 mPD-L1_shRNA
CCGAAATGATACACAATTCGA
>SEQ ID NO: 17 hPD-L1_shRNA_1
CCGGCGAATTACTGTGAAAGTCAAT
>SEQ ID NO: 18 hPD-L1_shRNA_2
CCGGCTGACATTCATCTTCCGTTTA
>SEQ ID NO: 19 hPD-L1_shRNA_1
CCGGCGAATTACTGTGAAAGTCAATCTCGAGATTGACTTTCACAGTAA
TTCGTTTTTG
>SEQ ID NO: 20 hPD-L1_shRNA_2
CCGGCTGACATTCATCTTCCGTTTACTCGAGTAAACGGAAGATGAATG
TCAGCCGGTTTTTG

### Example 1. Construction and In Vitro Activity Evaluation of Lentivirus Vectors for Knocking Out CMTM6 Expression

The inventors first designed sgRNA oligonucleotides with a length of 18-24 nucleotides targeting the CMTM6 coding sequence in the human or mouse genome through software. The actual matching results were verified by the NCBI database sequence alignment, with the preferred sequences being SEQ ID NOs: 13-15.

The sgRNA short gene sequences used to construct lentivirus vectors for knocking out CMTM6 expression were obtained by in vitro DNA chemical synthesis. The synthesized sgRNA short gene sequences were cloned into the lentiCRISPR lentivirus expression vector plasmid by enzyme digestion ligation, and the accuracy of the insertion sequences was verified by gene sequencing.

After validation, the lentivirus vector was transformed into E. coli Stb13 competent cells, and the vector plasmid was amplified and extracted. After purification and extraction of the lentivirus vector plasmid, the lentivirus vector plasmid and the virus packaging plasmid system (pSPAX2 and pMD2.G) were transfected into HEK293T cells.

Three days after the virus packaging in the HEK293T, PEG concentration reagent was shaken and concentrated overnight, and the lentivirus was concentrated and purified after high-speed centrifugation (4000×g, 30 min). The obtained lentivirus was a successful lentivirus vector that can be used to knock out CMTM6.

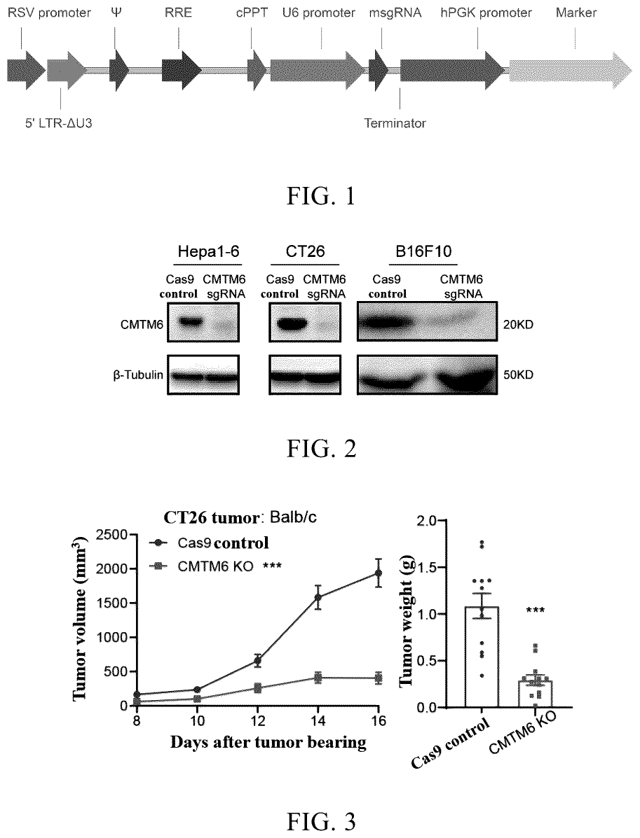

The coding pattern of the lentivirus for knocking out CMTM6 expression was shown in FIG. 1.

The above-obtained lentivirus solution was used to infect the following tumor cells cultured by in vitro adherent culture: Hepa1-6 liver cancer cells, CT26 colorectal cancer cells, and B16F10 melanoma cells. After virus infiltration and antibiotic screening, 1 million cells were counted and subjected to RIPA lysis and sonication to prepare the cell lysate. This lysate was then used for Western blot analysis to verify that the constructed lentivirus can be used for in vitro knockout of CMTM6 expression.

Using the classical Western blot method, polyacrylamide gel electrophoresis, wet transfer onto a membrane, blocking, incubation with the primary antibody, incubation with the secondary antibody, and exposure were sequentially carried out to obtain the final western blot exposure image, which was presented as FIG. 2.

The results shown in FIG. 2 indicated that the lentivirus constructed by the present invention could completely knock out CMTM6 expressed in Hepa1-6 liver cancer cells, CT26 colorectal cancer cells, and B16F10 melanoma cells in vitro, suggesting its high level of biological activity.

### Example 2. Evaluation of In Vivo Anti-Tumor Activity of Lentivirus Vectors for Knocking Out CMTM6 Expression

In order to evaluate the in vivo anti-tumor activity of the lentivirus vector for knocking out CMTM6 expression constructed by the present invention, the inventors evaluated its anti-tumor activity in a variety of xenograft models.

### (1) Evaluation of Anti-Tumor Activity Against CT26 Colorectal Cancer Tumors:

Resuscitate CT26 colorectal cancer tumor cells and passage them to ensure that the tumor cells have been passaged for at least 3 generations at the time of tumor bearing. CT26 tumor cells were inoculated subcutaneously into female BALB/c mice, and the inoculation dose was 5×10⁵ cells/mouse. The mice were randomly divided into 2 groups (10 mice in each group), namely: Cas9 control lentivirus treatment group (Cas9 control) and CMTM6 expression knockout lentivirus treatment group (CMTM6 KO).

The body weight of the mice as well as the length (L) and width (W) of the tumors were observed and recorded until the endpoint of dissection. The tumor volume (V) was calculated using the formula V=(L×W×W)/2, the tumor growth curves were plotted, and the tumor inhibition rates were calculated.

After the subcutaneous tumors of the mice had grown to a certain length, euthanasia was performed and the animals were dissected to take out the subcutaneous tumors. The tumor growth curves and endpoint tumor weights of mice were recorded.

As shown in FIG. 3, the results showed that in vivo, the lentivirus for knocking out CMTM6 expression of the present invention could significantly inhibit the tumor growth of CT26 colorectal cancer, with a tumor inhibition rate of up to 73%.

### (2) Evaluation of Anti-Tumor Activity Against MC38 Colorectal Cancer Tumors:

Resuscitate MC38 colorectal cancer tumor cells and passage them to ensure that the tumor cells have been passaged for at least 3 generations at the time of tumor bearing. MC38 tumor cells were inoculated subcutaneously into female C57BL/6 mice, and the inoculation dose was 5×10⁵ cells/mouse. The mice were randomly divided into 2 groups (10 mice in each group), namely: Cas9 control lentivirus treatment group (Cas9 control) and CMTM6 expression knockout lentivirus treatment group (CMTM6 KO).

The body weight of the mice as well as the length (L) and width (W) of the tumors were observed and recorded until the endpoint of dissection. The tumor volume (V) was calculated using the formula V=(L×W×W)/2, the tumor growth curves were plotted, and the tumor inhibition rates were calculated.

After the subcutaneous tumors of the mice had grown to a certain length, euthanasia was performed and the animals were dissected to take out the subcutaneous tumors. The tumor growth curves and endpoint tumor weights of mice were recorded.

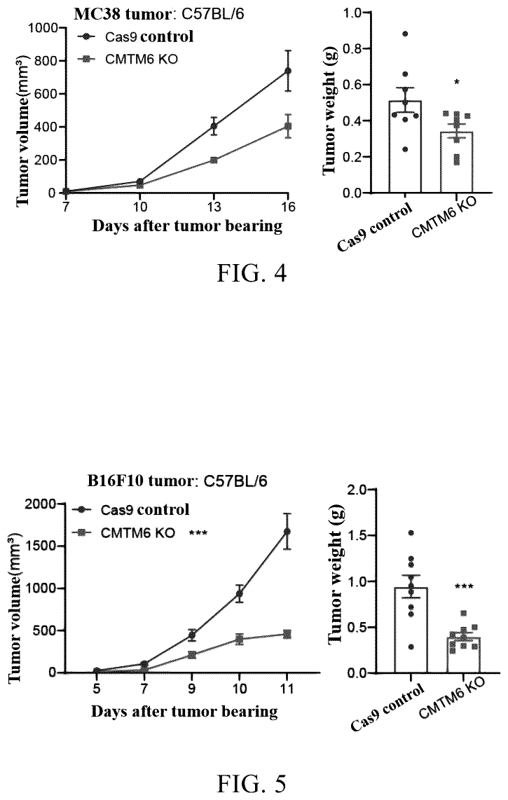

As shown in FIG. 4, the results showed that in vivo, the lentivirus for knocking out CMTM6 expression of the present invention could significantly inhibit the tumor growth of MC38 colorectal cancer, with a tumor inhibition rate of 33.4%.

### (3) Evaluation of Anti-Tumor Activity Against B16F10 Melanoma Tumors:

Resuscitate B16F10 melanoma tumor cells and passage them to ensure that the tumor cells have been passaged for at least 3 generations at the time of tumor bearing. B16F10 tumor cells were inoculated subcutaneously into female C57BL/6 mice, and the inoculation dose was 5×10⁵ cells/mouse. The mice were randomly divided into 2 groups (10 mice in each group), namely: Cas9 control lentivirus treatment group (Cas9 control) and CMTM6 expression knockout lentivirus treatment group (CMTM6 KO).

The body weight of the mice as well as the length (L) and width (W) of the tumors were observed and recorded until the endpoint of dissection. The tumor volume (V) was calculated using the formula V=(L×W×W)/2, the tumor growth curves were plotted, and the tumor inhibition rates were calculated.

After the subcutaneous tumors of the mice had grown to a certain length, euthanasia was performed and the animals were dissected to take out the subcutaneous tumors. The tumor growth curves and endpoint tumor weights of mice were recorded.

As shown in FIG. 5, the results showed that in vivo, the lentivirus for knocking out CMTM6 expression of the present invention could significantly inhibit the tumor growth of B16F10 melanoma, with a tumor inhibition rate of 57.8%.

### (4) Evaluation of Anti-Tumor Activity Against Hepa1-6 Liver Cancer Tumors:

Resuscitate Hepa1-6 liver cancer tumor cells and passage them to ensure that the tumor cells have been passaged for at least 3 generations at the time of tumor bearing. Hepa1-6 tumor cells were inoculated subcutaneously into female C57BL/6 mice, and the inoculation dose was 5×10⁵ cells/mouse. The mice were randomly divided into 2 groups (10 mice in each group), namely: Cas9 control lentivirus treatment group (Cas9 control) and CMTM6 expression knockout lentivirus treatment group (CMTM6 KO).

The body weight of the mice as well as the length (L) and width (W) of the tumors were observed and recorded until the endpoint of dissection. The tumor volume (V) was calculated using the formula V=(L×W×W)/2, the tumor growth curves were plotted, and the tumor inhibition rates were calculated.

After the subcutaneous tumors of the mice had grown to a certain length, euthanasia was performed and the animals were dissected to take out the subcutaneous tumors. The tumor growth curves and endpoint tumor weights of mice were recorded.

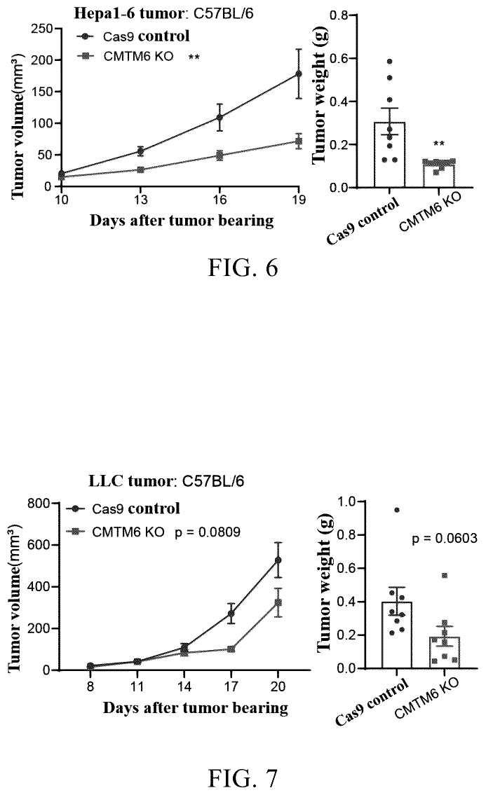

As shown in FIG. 6, the results showed that in vivo, the lentivirus for knocking out CMTM6 expression of the present invention could significantly inhibit the tumor growth of Hepa1-6 liver cancer, with a tumor inhibition rate of up to 64.4%.

### (5) Evaluation of Anti-Tumor Activity Against LLC Non-Small Cell Lung Cancer Tumors:

Resuscitate LLC non-small cell lung cancer tumor cells and passage them to ensure that the tumor cells have been passaged for at least 3 generations at the time of tumor bearing. LLC tumor cells were inoculated subcutaneously into female C57BL/6 mice, and the inoculation dose was 5×10⁵ cells/mouse. The mice were randomly divided into 2 groups (10 mice in each group), namely: Cas9 control lentivirus treatment group (Cas9 control) and CMTM6 expression knockout lentivirus treatment group (CMTM6 KO).

The body weight of the mice as well as the length (L) and width (W) of the tumors were observed and recorded until the endpoint of dissection. The tumor volume (V) was calculated using the formula V=(L×W×W)/2, the tumor growth curves were plotted, and the tumor inhibition rates were calculated.

After the subcutaneous tumors of the mice had grown to a certain length, euthanasia was performed and the animals were dissected to take out the subcutaneous tumors. The tumor growth curves and endpoint tumor weights of mice were recorded.

As shown in FIG. 7, the results showed that in vivo, the lentivirus for knocking out CMTM6 expression of the present invention could significantly inhibit the tumor growth of LLC non-small cell lung cancer, with a tumor inhibition rate of 52%.

The above results indicate that the lentivirus vector for knocking out CMTM6 expression of the present invention has excellent anti-tumor effects in vivo, which have been fully verified in colorectal cancer, melanoma, liver cancer, and non-small cell lung cancer, and its inhibition effect on tumor growth is excellent, showing good application prospects.

### Example 3. Evaluation of the Activity of Lentivirus Vectors for Knocking Out CMTM6 Expression Against PD-L1-Deficient Tumors

Existing research has shown that knocking out CMTM6 can reduce the expression of PD-L1 in tumors. However, many patients exhibit low or no expression of PD-L1 in clinical practice. Therefore, it is crucial to examine whether the lentivirus vector for knocking out CMTM6 expression of the present invention can mediate the in vivo growth inhibition of tumors with low or no PD-L1 expression.

On the one hand, in the Example 2 of the present invention, the inventors adopted a variety of xenograft tumor models that include CT26 and B16F10 two types of tumors with low expression of PD-L1, and the lentivirus vector for knocking out CMTM6 expression of the present invention could restrict tumor growth in vivo with extremely significant inhibitory effects of 73% and 57.8%, indicating that the lentivirus vector of the present invention is not affected by the low expression of PD-L1 in tumors.

Further, the inventors used PD-L1-deficient CT26 colorectal cancer tumor cells for subcutaneous tumor-bearing experiments. The results were shown in FIG. 9, indicating that:
1) As a control, the lentivirus vector for knocking out CMTM6 expression of the present invention demonstrated consistent tumor growth inhibition effects on regular CT26 cells as in Example 2;
2) As a control, the newly constructed lentivirus vector for knocking out PD-L1 expression of the present invention also exhibited excellent tumor inhibition effects on regular CT26 cells;
3) On the basis of PD-L1 deficiency in CT26 cells, the lentivirus vector for knocking out CMTM6 expression of the present invention displayed better anti-tumor effects. Specifically, the tumor inhibition rate was 100%, and all tumor-bearing mice showed tumor regression, indicating unexpected anti-tumor effects with a tumor regression rate of 100%.

The above results show that the lentivirus vector for knocking out CMTM6 expression of the present invention still has excellent anti-tumor effects on tumors with no expression of PD-L1.

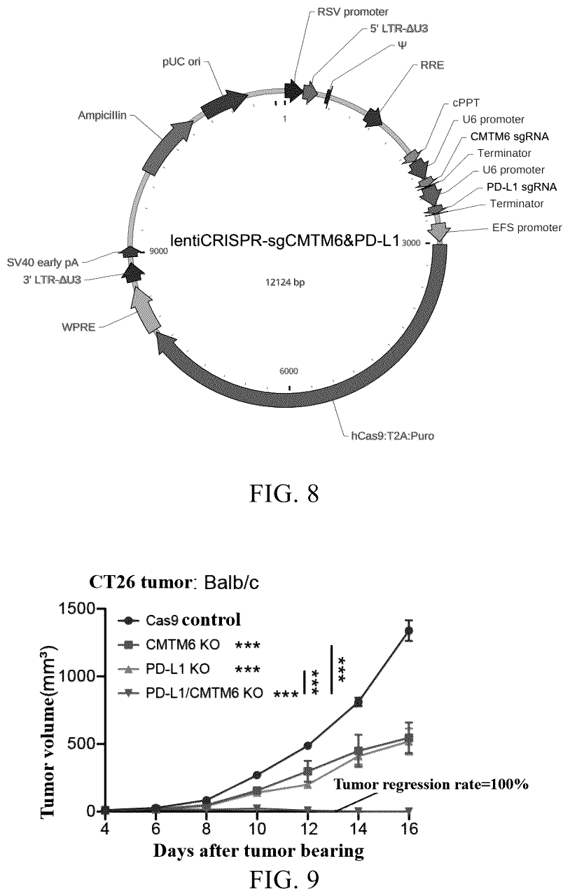

On this basis, a lentivirus vector for simultaneously knocking out CMTM6 and PD-L1 was also constructed by the present invention, and its coding pattern was shown in FIG. 8.

Using the newly constructed lentivirus vector for simultaneously knocking out CMTM6 and PD-L1 of the present invention for the treatment of CT26 colorectal cancer tumors, consistent results were found with the above descriptions, showing a tumor regression rate of 100%.

In order to further verify whether such anti-tumor effects have immune memory, the inventors re-challenged the surviving mice with tumor regression by inoculating CT26 colorectal cancer cells on the opposite limb, and then observed and recorded the tumor growth.

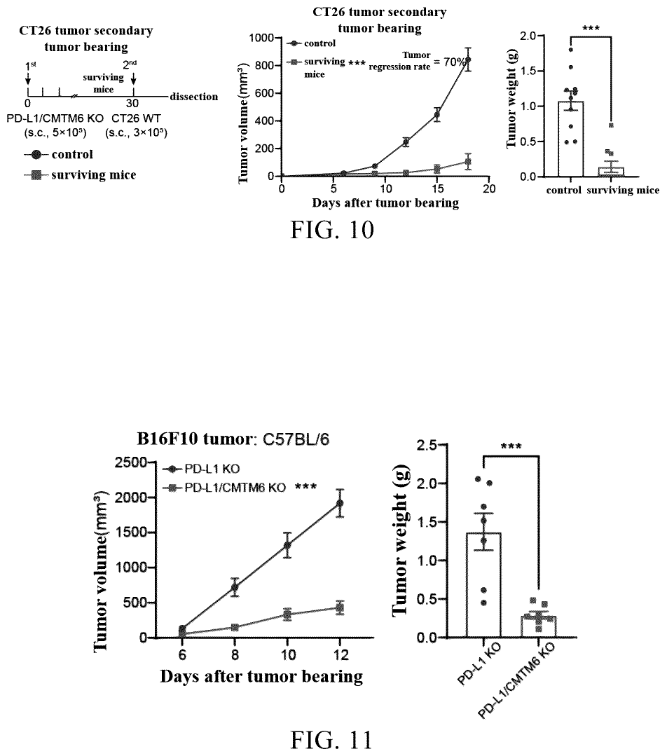

As shown in FIG. 10, the results showed that the surviving mice with tumor regression after treatment with the lentivirus vector for simultaneously knocking out CMTM6 and PD-L1 exhibited a surprising anti-tumor immune memory against CT26 colorectal cancer tumors. The tumor regression rate upon re-challenge remained as high as 70%, and the volume and size of the non-regressed tumors were also significantly smaller than those in the control group of mice.

The above indicates that the lentivirus vector of the present invention not only has excellent anti-tumor efficacy, but also can stimulate anti-tumor immune memory in vivo.

Similarly, the inventors used a xenograft model of B16F10 melanoma to test the therapeutic effects of the lentivirus vector for simultaneously knocking out CMTM6 and PD-L1, observing tumor growth curves and statistical analysis of tumor weight at the endpoint dissection during the experimental period.

As shown in FIG. 11, the lentivirus vector had significant anti-tumor effects, and the effects were significantly better than the lentivirus vector for knocking out PD-L1 constructed by the present invention.

To further test the efficacy of the lentivirus vector for knocking out CMTM6 against tumors with PD-1/PD-L1 axis deficiency, the inventors used a xenograft model of MC38 colorectal cancer. MC38 colorectal cancer is a type of tumor that is not sensitive to PD-L1 deficiency, meaning that knocking out PD-L1 in MC38 has no significant impact on its in vivo growth.

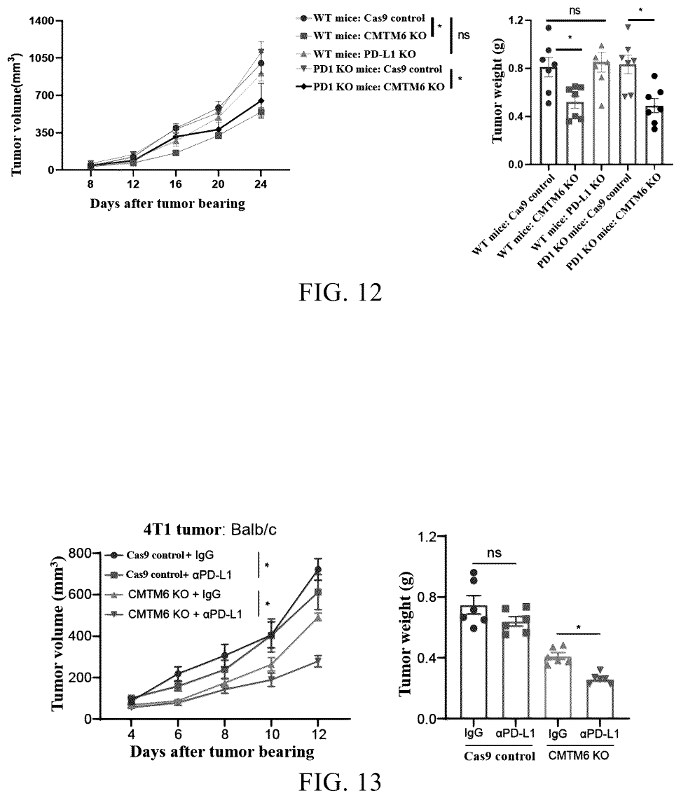

According to the experimental results of the present invention, as shown in FIG. 12, in wild-type mice, there was no difference in tumor growth between the Cas9 control group and the PD-L1 KO group, which is consistent with existing research foundations, demonstrating the reliability of our experimental system.

Then, in wild-type mice, when comparing the Cas9 control group with the CMTM6 KO group, surprisingly, the lentivirus vector for knocking out CMTM6 of the present invention showed significant effects, indicating that the lentivirus vector for knocking out CMTM6 of the present invention can effectively inhibit tumors that are unresponsive to PD-L1 deficiency.

On the other hand, in PD-1-deficient mice, PD-1 deficiency also had no significant effect on tumor growth. However, when comparing the Cas9 control group with the CMTM6 KO group at this point, it was again found that the lentivirus vector for knocking out CMTM6 expression of the present invention exhibited significant effects.

The above indicates that the lentivirus vector of the present invention still has good anti-tumor effects on PD-1/PD-L1 deficient tumors, and still has inhibitory effects on tumors that are unresponsive to PD-L1 deficiency.

### Example 4. Evaluation of the Activity of Lentivirus Vectors for Knocking Out CMTM6 Expression Against Tumors Resistant to Immune Checkpoint Antibodies

Immune checkpoint antibody therapy, represented by PD-1/PD-L1/CTLA-4 antibodies, has shown promising results in various tumor treatments. However, there are still many tumors that are unresponsive to the immune checkpoint antibody therapy. For these non-responsive tumors, combination therapies are often needed to improve response rates and achieve therapeutic effects. Therefore, the inventors of the present invention utilized two tumor models that are resistant to immune checkpoint antibody therapy: the 4T1 breast cancer xenograft model and the LLC non-small cell lung cancer xenograft model, to test whether the lentivirus vector for knocking out CMTM6 expression of the present invention can overcome the resistance and non-responsiveness of tumors to the immune checkpoint antibody therapy.

### (1) 4T1 Breast Cancer Xenograft Model:

Resuscitate 4T1 breast cancer tumor cells and passage them to ensure that the tumor cells have been passaged for at least 3 generations at the time of tumor bearing. 4T1 tumor cells were inoculated subcutaneously into female BALB/c mice, and the inoculation dose was 5×10⁵ cells/mouse. The mice were randomly divided, namely: Cas9 control lentivirus+IgG administration group (Cas9 control+IgG), Cas9 control lentivirus+PD-L1 antibody administration group (Cas9 control+αPD-L1), lentivirus for knocking out CMTM6+IgG administration group (CMTM6 KO+IgG), and lentivirus for knocking out CMTM6+PD-L1 antibody administration group (CMTM6 KO+αPD-L1).

During the dosing procedure, the body weight of the mice as well as the length (L) and width (W) of the tumors were observed and recorded until the endpoint of dissection. The tumor volume (V) was calculated using the formula V=(L×W×W)/2, the tumor growth curves were plotted, and the tumor inhibition rates were calculated.

After the subcutaneous tumors of the mice had grown to a certain length, euthanasia was performed and the animals were dissected to take out the subcutaneous tumors. The tumor growth curves and endpoint tumor weights of mice were recorded.

As shown in FIG. 13, the results showed that the PD-L1 antibody did not have significant inhibitory effects on 4T1 tumors, and the lentivirus vector for knocking out CMTM6 expression of the present invention could significantly inhibit the growth of 4T1 tumors. Surprisingly, the PD-L1 antibody combined with the lentivirus vector of the present invention could better and significantly antagonize the in vivo growth of 4T1, meaning that the lentivirus vector for knocking out CMTM6 expression of the present invention can respond to immune checkpoint-resistant tumors, and improve the response of immune checkpoint-resistant/-non-responsive tumors to checkpoint antibodies.

### (2) LLC Non-Small Cell Lung Cancer Xenograft Model:

Similarly, resuscitate LLC non-small cell lung cancer tumor cells and passage them to ensure that the tumor cells have been passaged for at least 3 generations at the time of tumor bearing. LLC tumor cells were inoculated subcutaneously into female C57BL/6 mice, and the inoculation dose was 5×10⁵ cells/mouse. The mice were randomly divided, namely: Cas9 control lentivirus+IgG administration group (Cas9 control+IgG), Cas9 control lentivirus+PD-L1 antibody administration group (Cas9 control+αPD-L1), Cas9 control lentivirus+CTLA-4 antibody administration group (Cas9 control+αCTLA-4), lentivirus for knocking out CMTM6+IgG administration group (CMTM6 KO+IgG), lentivirus for knocking out CMTM6+PD-L1 antibody administration group (CMTM6 KO+αPD-L1), and lentivirus for knocking out CMTM6+CTLA-4 antibody administration group (CMTM6 KO+αCTLA-4).

During the dosing procedure, the body weight of the mice as well as the length (L) and width (W) of the tumors were observed and recorded until the endpoint of dissection. The tumor volume (V) was calculated using the formula V=(L×W×W)/2, the tumor growth curves were plotted, and the tumor inhibition rates were calculated.

After the subcutaneous tumors of the mice had grown to a certain length, euthanasia was performed and the animals were dissected to take out the subcutaneous tumors. The tumor growth curves and endpoint tumor weights of mice were recorded.

As shown in FIG. 14, the results showed that PD-L1 antibody and CTLA-4 antibody did not have significant inhibitory effects on LLC tumors, and the lentivirus vector for knocking out CMTM6 expression of the present invention could significantly inhibit the growth of LLC tumors. Surprisingly, the combination of PD-L1 antibody or CTLA-4 antibody with the lentivirus vector of the present invention could better and significantly antagonize the in vivo growth of LLC, meaning that the lentivirus vector for knocking out CMTM6 expression of the present invention can respond to immune checkpoint-resistant tumors, and improve the response of immune checkpoint-resistant/-non-responsive tumors to checkpoint antibodies.

### Example 5. Evaluation of the Anti-Tumor Metastasis Activity of Lentivirus Vectors for Knocking Out CMTM6 Expression

Tumor cells metastasize from the primary site to the secondary site via lymphatic vessels, blood vessels, or adjacent tissues, which is often the cause of treatment failure for malignant tumors. Therefore, preventing or limiting the metastasis of tumor cells in tumor immunotherapy is an urgent concern. The inventors used a lung metastasis model of B16F10 melanoma and a lung metastasis model of 4T1 breast cancer to evaluate the effects of the lentivirus vector for knocking out CMTM6 expression of the present invention on restricting tumor metastasis.

### (1) Lung Metastasis Model of B16F10 Melanoma:

Resuscitate B16F10 melanoma tumor cells and passage them to ensure that the tumor cells have been passaged for at least 3 generations at the time of tumor bearing. B16F10 tumor cells were injected into female C57BL/6 mice via tail vein injection, and the cells were treated with Cas9 control lentivirus vector or the lentivirus vector for knocking out CMTM6 expression of the present invention, with a cell dose of 1×10⁶ and a cell suspension volume of 200 μL.

After 25 days of tumor growth, the mice were dissected, and the whole lung tissues were taken out, weighed and photographed.

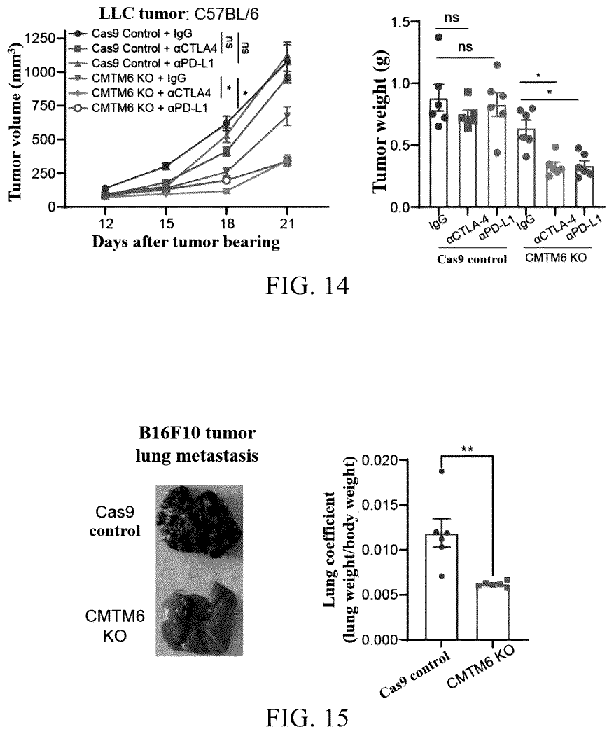

According to the statistical results of the lung photos and lung coefficients (the ratios of mouse lung weights to mouse body weights) at the dissection endpoint, as shown in FIG. 15, the lungs of mice in the control group had been covered with melanoma metastases, while after the treatment with the lentivirus vector for knocking out CMTM6 expression of the present invention, only a few melanoma metastases were found in the lungs of mice. Meanwhile, the lung coefficients were also significantly lower than those of the control group, indicating that the pathological damage of the lungs was significantly lower than that of the control group.

The above indicates that the lentivirus vector for knocking out CMTM6 expression of the present invention can limit lung metastasis of melanoma.

### (2) Lung Metastasis Model of 4T1 Breast Cancer:

Resuscitate 4T1 breast cancer tumor cells and passage them to ensure that the tumor cells have been passaged for at least 3 generations at the time of tumor bearing. 4T1 tumor cells were injected into female BALB/c mice via tail vein injection, and the cells were treated with Cas9 control lentivirus vector or the lentivirus vector for knocking out CMTM6 expression of the present invention, with a cell dose of 1×10⁶ and a cell suspension volume of 200 μL.

After 25 days of tumor growth, the mice were dissected, and the whole lung tissues were taken out, weighed and photographed.

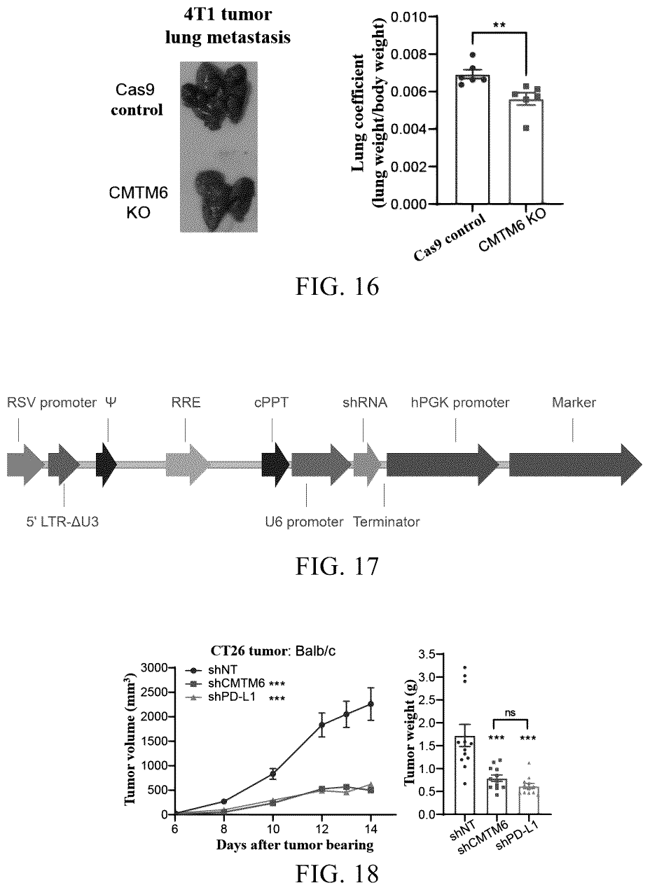

According to the statistical results of lung photos and lung coefficients (the ratios of mouse lung weights to mouse body weights) at the dissection endpoint, as shown in FIG. 16, the lungs of mice in the control group had been covered with breast cancer tumor metastases, while after the treatment with the lentivirus vector for knocking out CMTM6 expression of the present invention, only a few breast cancer tumor metastases were found in the lungs of mice. Meanwhile, the lung coefficients were also significantly lower than those of the control group, indicating that the pathological damage of the lungs was significantly lower than that of the control group.

The above indicates that the lentivirus vector for knocking out CMTM6 expression of the present invention can limit lung metastasis of breast cancer tumors.

In summary, in both tumor metastasis models, the lentivirus vector for knocking out CMTM6 expression of the present invention exhibited excellent inhibition on tumor metastasis, indicating its application in tumor immunotherapy to prevent or limit tumor metastasis.

### Example 6. Construction of Lentivirus Vectors for Interfering with CMTM6 Expression and Evaluation of Anti-Tumor Activity In Vivo

Examples 1-5 have shown the in vitro and in vivo anti-tumor activity of the lentivirus vector for knocking out CMTM6 expression of the present invention, and the inventors have also constructed a lentivirus vector for interfering with CMTM6 expression based on a short hairpin RNA (shRNA) oligonucleotide, to achieve the degradation of CMTM6 transcription RNA to interfere with the expression of CMTM6.

The inventors first designed shRNA oligonucleotides with a length of 18-24 nucleotides targeting the CMTM6 coding sequence in the human or mouse genome through software. The actual matching results were verified by the NCBI database sequence alignment, with the preferred sequences being SEQ ID NOs: 1-12.

The shRNA short gene sequences used to construct lentivirus vectors for interfering with CMTM6 expression were obtained by in vitro DNA chemical synthesis. The synthesized shRNA short gene sequences were cloned into the pLKO.1 lentivirus expression vector plasmid by enzyme digestion ligation, and the accuracy of the insertion sequences was verified by gene sequencing.

After validation, the lentivirus vector was transformed into E. coli Stb13 competent cells, and the vector plasmid was amplified and extracted. After purification and extraction of the lentivirus vector plasmid, the lentivirus vector plasmid and the virus packaging plasmid system (pSPAX2 and pMD2.G) were transfected into HEK293T cells.

Three days after the virus packaging in the HEK293T, PEG concentration reagent was shaken and concentrated overnight, and the lentivirus was concentrated and purified after high-speed centrifugation (4000×g, 30 min). The obtained lentivirus was a successful lentivirus vector that can be used to interfere with CMTM6.

The coding pattern of the lentivirus for interfering with CMTM6 expression was shown in FIG. 17.

At the same time, the present invention also constructed a lentivirus vector for interfering with PD-L1 expression, and the construction process was the same as above.

In order to evaluate the in vivo anti-tumor activity of the lentivirus vector for interfering with CMTM6 expression constructed by the present invention, the inventors used the CT26 colorectal cancer xenograft model to evaluate its anti-tumor activity.

Resuscitate CT26 colorectal cancer tumor cells and passage them to ensure that the tumor cells have been passaged for at least 3 generations at the time of tumor bearing. CT26 tumor cells were inoculated subcutaneously into female BALB/c mice, and the inoculation dose was 5×10⁵ cells/mouse. The mice were randomly divided into 3 groups (12 mice in each group), namely: irrelevant shRNA lentivirus treatment group (shNT), lentivirus interfering with CMTM6 expression treatment group (shCMTM6), and lentivirus interfering with PD-L1 expression treatment group (shPD-L1).

The body weight of the mice as well as the length (L) and width (W) of the tumors were observed and recorded until the endpoint of dissection. The tumor volume (V) was calculated using the formula V=(L×W×W)/2, the tumor growth curves were plotted, and the tumor inhibition rates were calculated.

After the subcutaneous tumors of the mice had grown to a certain length, euthanasia was performed and the animals were dissected to take out the subcutaneous tumors. The tumor growth curves and endpoint tumor weights of mice were recorded.

As shown in FIG. 18, the results showed that in vivo, the lentivirus for interfering with CMTM6 expression of the present invention could significantly inhibit the tumor growth of CT26 colorectal cancer, with a tumor inhibition rate of up to 74%, which was comparable to the effect of the lentivirus vector for interfering with PD-L1 expression.

The above indicates that the lentivirus vector for interfering with CMTM6 expression constructed by the present invention has excellent anti-tumor activity in vivo.

### Example 7. Construction of Adeno-Associated Virus Vectors that Interfere with and Knock Out CMTM6 Expression

In addition to using lentivirus as a vector for gene therapy to knock out or interfere with CMTM6 expression to inhibit tumor growth, the inventors also constructed a gene therapy method using adeno-associated virus (AAV) as a vector to interfere with and knock out CMTM6 expression.

The inventors first designed shRNA oligonucleotides with a length of 18-24 nucleotides targeting the CMTM6 coding sequence in the human or mouse genome through software. The actual matching results were verified by the NCBI database sequence alignment, with the preferred sequences being SEQ ID NOs: 1-12. Alternatively, the inventors designed sgRNA oligonucleotides with a length of 18-24 nucleotides targeting the CMTM6 coding sequence in the human or mouse genome through software. The actual matching results were verified by the NCBI database sequence alignment, with the preferred sequences being SEQ ID NOs: 13-15.

The short gene sequences used to construct adeno-associated virus vectors for interfering with or knocking out CMTM6 expression were obtained by in vitro DNA chemical synthesis. The synthesized shRNA/sgRNA short gene sequences were cloned into the pscAAV-EGFP-shRNA vector plasmid by enzyme digestion ligation, and the accuracy of the insertion sequences was verified by gene sequencing.

After validation, the pscAAV-EGFP-shRNA vector was transformed into E. coli DH5a competent cells, and the vector plasmid was amplified and extracted. After purification and extraction of the vector plasmid, the pscAAV-EGFP-shRNA vector plasmid and the virus packaging plasmid system (pAAV-RC9 and pHelper) were transfected into HEK293T-AAV cells.

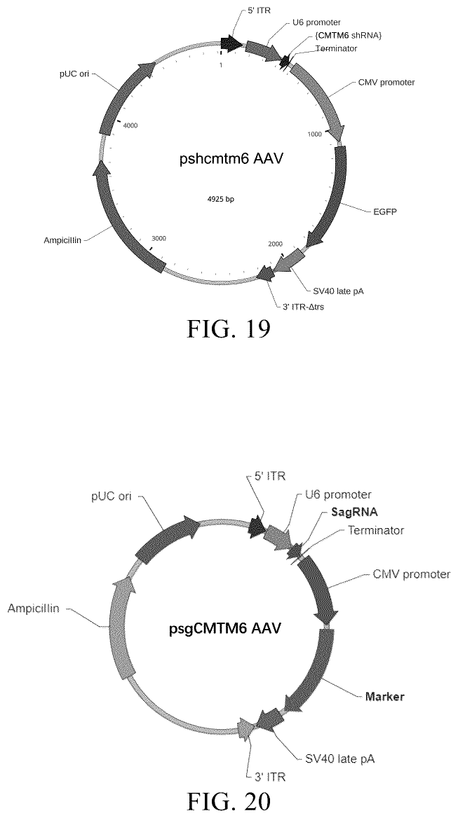

Three days after virus packaging in HEK293T-AAV, the concentrated and purified adeno-associated viruses were obtained using a concentrated reagent. The obtained viruses were successful adeno-associated virus vectors that could be used to interfere with or knock out CMTM6, and their genome coding patterns were shown in FIG. 19 and FIG. 20, respectively.

### Example 8. In Vitro Evaluation of Adeno-Associated Virus Vectors for Interfering with CMTM6 Expression

The inventors have evaluated the ability of constructed adeno-associated virus vectors that interfere with CMTM6 expression to invade tumor cells and reduce CMTM6 expression in tumor cells in vitro.

The inventors inoculated CT26 and B16F10 cells in 24-well plates at a cell density of 3×10⁵ cells/mL at 400 μL per well. After the cells were adhered overnight, normal saline control, control shRNA adeno-associated virus (shNC AAV) and adeno-associated virus interfering with CMTM6 expression (shCMTM6 AAV) were added, and the infiltration MOI of adeno-associated virus was 1:10000. After 48 hours of virus infiltration, cells were taken to evaluate the in vitro activity of the adeno-associated virus vector interfering with CMTM6 expression of the present invention at the levels of flow cytometry, immunofluorescence and Western blot.

After trypsin digestion of the adherent cells and two washes with PBS buffer, the cells were resuspended into a single-cell suspension and loaded onto a flow cytometer for detection. The FITC fluorescence channel was selected to detect the EGFP protein expressed in the cells.

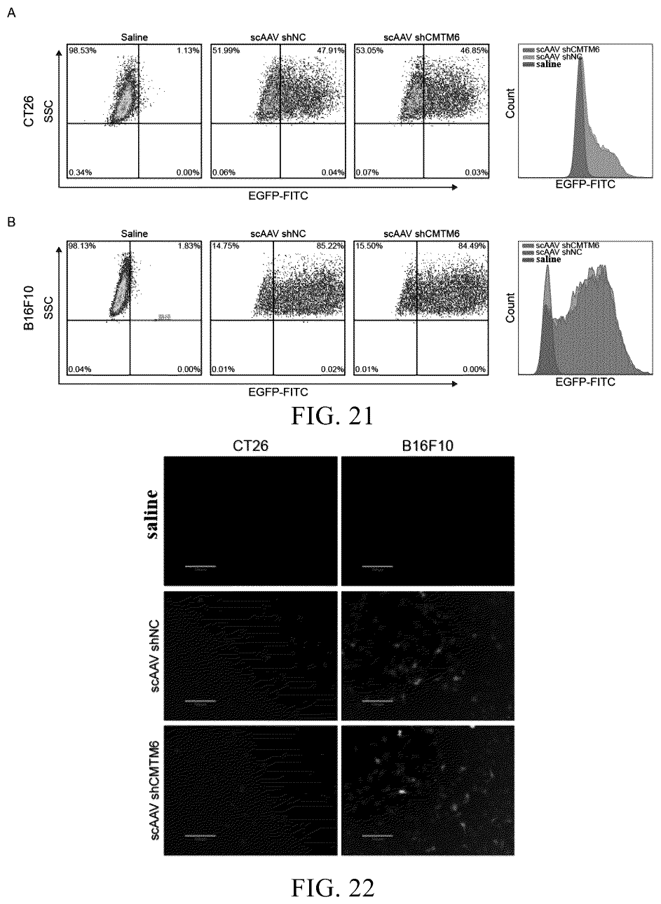

From the flow cytometry results, as shown in FIG. 21, it can be seen that in vitro, the adeno-associated virus of the present invention can effectively enter tumor cells, with an infection efficiency of 46.85% in CT26 and up to 84.49% in B16F10 cells.

After the adherent cells were washed twice with PBS buffer, the cover of the cell culture plate was removed, and the plate was placed under an inverted fluorescence microscope. After determining the field of view in a bright field microscope, a FITC fluorescent lens was selected to take several photos.

From the results of immunofluorescence, as shown in FIG. 22, similarly, the adeno-associated virus of the present invention effectively entered CT26 and B16F10 tumor cells and efficiently expressed EGFP green fluorescent protein in the tumor cells.

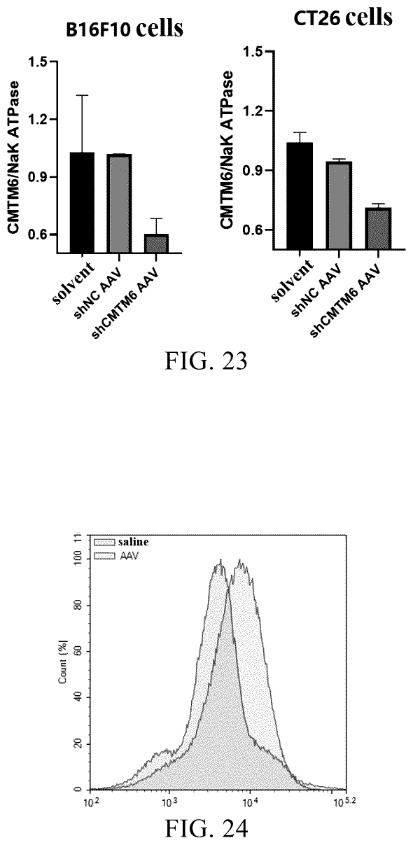

In order to evaluate the ability of adeno-associated virus to reduce the expression of CMTM6 in tumor cells in vitro, the inventors extracted total protein and prepared immunoblot samples from infected CT26 and B16F10 cells. Polyacrylamide gel electrophoresis, transfer onto a membrane, incubation with the primary antibody and secondary antibody and exposure were carried out, and Na+K ATPase was selected as the membrane protein internal control. As shown in FIG. 23, it was found that the adeno-associated virus of the present invention could significantly interfere with CMTM6 expression.

The above results indicate that the adeno-associated virus vector that interferes with the expression of CMTM6 constructed by the present invention has good activity in invading tumor cells, and can effectively reduce the expression of CMTM6 in tumor cells in vitro.

### Example 9. Evaluation of the In Vitro Distribution of Adeno-Associated Virus Vectors for Interfering with CMTM6 Expression

The inventors have confirmed that the constructed adeno-associated virus that interferes with the expression of CMTM6 can infect tumor cells in vitro, and have also verified the infection effects in vivo.

Resuscitate CT26 colorectal cancer tumor cells and passage them to ensure that the tumor cells have been passaged for at least 3 generations at the time of tumor bearing. CT26 tumor cells were inoculated subcutaneously into female BALB/c mice, and the inoculation dose was 5×10⁵ cells/mouse. After the tumor volume grew to 200 mm³, 1×10¹⁰ vg of the adeno-associated virus of the present invention was injected peritumorally.

After 4 days, the mice were dissected, the tumor tissues were taken and the tissues were digested with type IV collagenase and hyaluronidase. After filtering the cell digestion solution through a 200 μm cell sieve and washing twice with PBS buffer, the red blood cells in the tissue cell suspension were removed using red blood cell lysis buffer. Single-cell Fc receptors were blocked using mouse Fc blockers.

Finally, the obtained single-cell suspension was stained and labeled with fluorescent antibodies, and the EGFP fluorescence signal level of CD45-negative tumor cells was detected by flow cytometry.

As shown in FIG. 24, within the tumor injected with the adeno-associated virus of the present invention peritumorally, tumor cells expressed high levels of EGFP, indicating that the adeno-associated virus of the present invention had good in vivo infectious activity.

### Example 10. Evaluation of Anti-Tumor Activity of Adeno-Associated Virus Vectors for Interfering with CMTM6 Expression

In order to evaluate the in vivo anti-tumor activity of the adeno-associated virus vector for interfering with CMTM6 expression constructed by the present invention, the inventors evaluated its anti-tumor activity in a variety of xenograft models.

### (1) Evaluation of Anti-Tumor Activity Against CT26 Colorectal Cancer Tumors:

Resuscitate CT26 colorectal cancer tumor cells and passage them to ensure that the tumor cells have been passaged for at least 3 generations at the time of tumor bearing. CT26 tumor cells were inoculated subcutaneously into female BALB/c mice, and the inoculation dose was 5×10⁵ cells/mouse. The mice were randomly divided into 2 groups, namely: control shRNA adeno-associated virus treatment group (shNC scAAV9) and adeno-associated virus vector interfering with CMTM6 expression treatment group (shCMTM6 scAAV9).

The body weight of the mice as well as the length (L) and width (W) of the tumors were observed and recorded until the endpoint of dissection. The tumor volume (V) was calculated using the formula V=(L×W×W)/2, the tumor growth curves were plotted, and the tumor inhibition rates were calculated.

After the subcutaneous tumors of the mice had grown to a certain length, euthanasia was performed and the animals were dissected to take out the subcutaneous tumors. The tumor growth curves and endpoint tumor weights of mice were recorded.

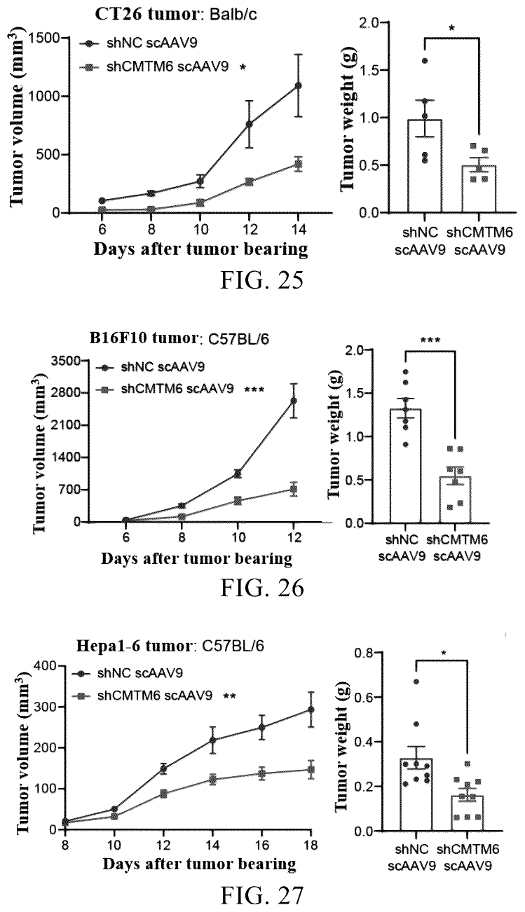

As shown in FIG. 25, the results showed that in vivo, the adeno-associated virus vector for interfering with CMTM6 expression of the present invention could significantly inhibit the tumor growth of CT26 colorectal cancer, with a tumor inhibition rate of 48.9%.

### (2) Evaluation of Anti-Tumor Activity Against B16F10 Melanoma Tumors:

Resuscitate B16F10 melanoma tumor cells and passage them to ensure that the tumor cells have been passaged for at least 3 generations at the time of tumor bearing. B16F10 tumor cells were inoculated subcutaneously into female C57BL/6 mice, and the inoculation dose was 5×10⁵ cells/mouse. The mice were randomly divided into 2 groups, namely: control shRNA adeno-associated virus treatment group (shNC scAAV9) and adeno-associated virus vector interfering with CMTM6 expression treatment group (shCMTM6 scAAV9).

The body weight of the mice as well as the length (L) and width (W) of the tumors were observed and recorded until the endpoint of dissection. The tumor volume (V) was calculated using the formula V=(L×W×W)/2, the tumor growth curves were plotted, and the tumor inhibition rates were calculated.

After the subcutaneous tumors of the mice had grown to a certain length, euthanasia was performed and the animals were dissected to take out the subcutaneous tumors. The tumor growth curves and endpoint tumor weights of mice were recorded.

As shown in FIG. 26, the results showed that in vivo, the adeno-associated virus vector for interfering with CMTM6 expression of the present invention could significantly inhibit the tumor growth of B16F10 melanoma, with a tumor inhibition rate of 58.8%.

### (3) Evaluation of Anti-Tumor Activity Against Hepa1-6 Liver Cancer Tumors:

Resuscitate Hepa1-6 liver cancer tumor cells and passage them to ensure that the tumor cells have been passaged for at least 3 generations at the time of tumor bearing. Hepa1-6 tumor cells were inoculated subcutaneously into female C57BL/6 mice, and the inoculation dose was 5×10⁵ cells/mouse. The mice were randomly divided into 2 groups, namely: control shRNA adeno-associated virus treatment group (shNC scAAV9) and adeno-associated virus vector interfering with CMTM6 expression treatment group (shCMTM6 scAAV9).

The body weight of the mice as well as the length (L) and width (W) of the tumors were observed and recorded until the endpoint of dissection. The tumor volume (V) was calculated using the formula V=(L×W×W)/2, the tumor growth curves were plotted, and the tumor inhibition rates were calculated.

After the subcutaneous tumors of the mice had grown to a certain length, euthanasia was performed and the animals were dissected to take out the subcutaneous tumors. The tumor growth curves and endpoint tumor weights of mice were recorded.

As shown in FIG. 27, the results showed that in vivo, the adeno-associated virus vector for interfering with CMTM6 expression of the present invention could significantly inhibit the tumor growth of Hepa1-6 liver cancer, with a tumor inhibition rate of up to 78.8%.

The above results indicate that the adeno-associated virus vector for interfering with CMTM6 expression of the present invention has excellent anti-tumor effects in vivo, which have been fully verified in colorectal cancer, melanoma, and liver cancer, and its inhibition effect on tumor growth is excellent, showing good application prospects.

### Example 11. Construction of Adeno-Associated Virus Vectors for Simultaneously Interfering with CMTM6 and PD-L1 Expressions and Evaluation of Anti-Tumor Activity

The inventors have also constructed gene therapies that use adeno-associated viruses as vectors to simultaneously interfere with the expression of CMTM6 and PD-L1.

The inventors first designed shRNA oligonucleotides with a length of 18-24 nucleotides targeting the RNA sequences of CMTM6 and PD-L1 in the human or mouse transcriptome through software. The actual matching results were verified by the NCBI database sequence alignment, with the sequences targeting CMTM6 preferably being SEQ ID NOs: 1-15, and the sequences targeting PD-L1 preferably being SEQ ID NOs: 16-20.

The short gene sequences used to construct adeno-associated virus vectors were obtained by in vitro DNA chemical synthesis. The synthesized shRNA short gene sequences were cloned into the pscAAV-EGFP-shRNA2 vector plasmid by enzyme digestion ligation, and the accuracy of the insertion sequence was verified by gene sequencing.

After validation, the pscAAV-EGFP-shRNA2 vector was transformed into E. coli DH5a competent cells, and the vector plasmid was amplified and extracted. After purification and extraction of the vector plasmid, the pscAAV-EGFP-shRNA2 vector plasmid and the virus packaging plasmid system (pAAV-RC9 and pHelper) were transfected into HEK293T-AAV cells.

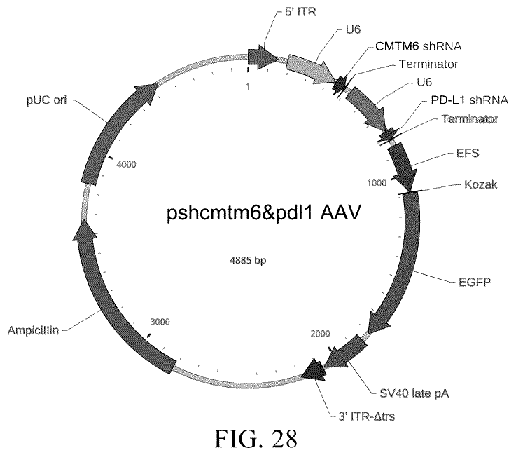

Three days after the virus packaging in HEK293T-AAV, the concentrated and purified adeno-associated virus was obtained by using a concentrated reagent. The obtained virus was a successful adeno-associated virus vector that could be used to interfere with the expression of CMTM6 and PD-L1 simultaneously, and its genome coding pattern was shown in FIG. 28.

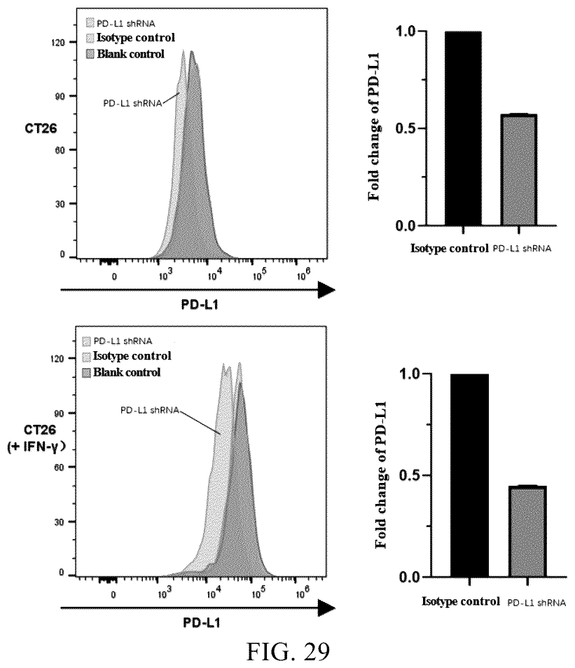

The foregoing examples of the present invention have evaluated the effects of shRNA interfering with CMTM6 expression. Here, at the level of tumor cells in vitro, adeno-associated viruses interfering with PD-L1 expression were expressed by infiltration. The PD-L1 level on the surface of colorectal cancer cell CT26 was analyzed by flow cytometry. As shown in FIG. 29, it was found that the shRNA or adeno-associated virus targeting PD-L1 of the present invention could effectively reduce PD-L1 expression, even in the presence of interferon stimulated PD-L1 expression.

In order to evaluate the in vivo anti-tumor activity of the adeno-associated virus vector for simultaneously interfering with CMTM6 and PD-L1 expressions constructed by the present invention, the inventors evaluated its anti-tumor activity in the CT26 colorectal cancer xenograft model.

Resuscitate CT26 colorectal cancer tumor cells and passage them to ensure that the tumor cells have been passaged for at least 3 generations at the time of tumor bearing. CT26 tumor cells were inoculated subcutaneously into female BALB/c mice, and the inoculation dose was 5×10⁵ cells/mouse. The mice were randomly divided into 4 groups, namely: control shRNA adeno-associated virus treatment group (shNC scAAV) and adeno-associated virus vector interfering with CMTM6 expression treatment group (shCMTM6 scAAV); adeno-associated virus vector interfering with PD-L1 expression treatment group (shPD-L1 scAAV); adeno-associated virus vector simultaneously interfering with CMTM6 and PD-L1 expression treatment group (shCMTM6&PD-L1 scAAV). The administration dosages were all 1× 10¹⁰ vg.

The body weight of the mice as well as the length (L) and width (W) of the tumors were observed and recorded until the endpoint of dissection. The tumor volume (V) was calculated using the formula V=(L×W×W)/2, the tumor growth curves were plotted, and the tumor inhibition rates were calculated.

After the subcutaneous tumors of the mice had grown to a certain length, euthanasia was performed and the animals were dissected to take out the subcutaneous tumors. The tumor growth curves and endpoint tumor weights of mice were recorded.

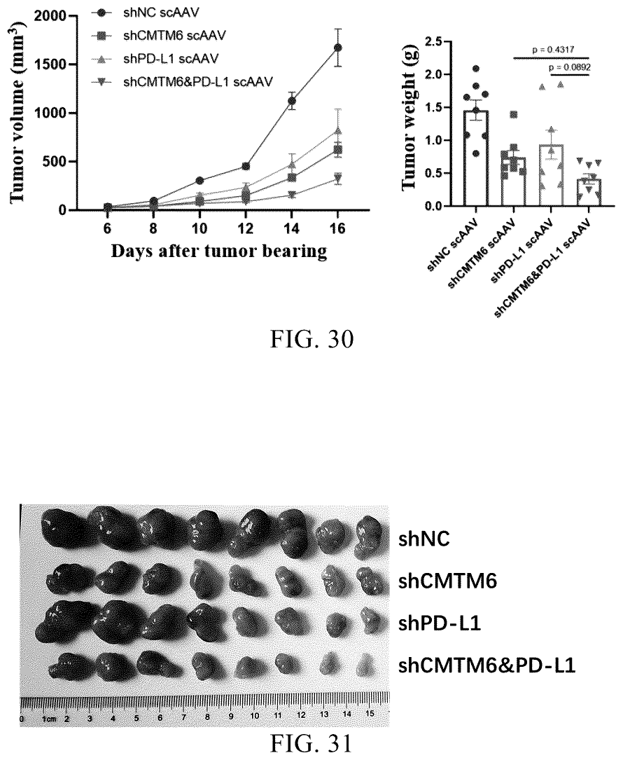

As shown in FIG. 30 and FIG. 31, the results showed that in vivo, the adeno-associated virus vector for interfering with CMTM6 expression of the present invention could still significantly inhibit the tumor growth of CT26 colorectal cancer, and its anti-tumor effects were relatively better than those of the adeno-associated virus vector for interfering with PD-L1 expression. In addition, the adeno-associated virus vector for simultaneously interfering with CMTM6 and PD-L1 expressions constructed by the present invention showed significantly better effects than those of the adeno-associated virus for interfering with CMTM6 expression alone and for interfering with PD-L1 expression alone.

The above indicates that the adeno-associated virus for simultaneously interfering with CMTM6 and PD-L1 expressions of the present invention has excellent anti-tumor effects, and its application value is even higher than that of the adeno-associated virus for interfering with CMTM6 expression.

### Example 12. Evaluation of Anti-Tumor Activity of Multiple Combination Therapy Regimens of Adeno-Associated Virus Vectors Interfering with CMTM6 Expression

The inventors also evaluated the combination therapy regimens and effects of adeno-associated virus vectors interfering with CMTM6 expression in combination with immune checkpoint antibodies, immune agonists, chemotherapy drugs, lipid metabolism regulating drugs, and glucose metabolism regulating drugs.

Resuscitate CT26 colorectal cancer tumor cells and passage them to ensure that the tumor cells have been passaged for at least 3 generations at the time of tumor bearing. CT26 tumor cells were inoculated subcutaneously into female BALB/c mice, and the inoculation dose was 5×10⁵ cells/mouse. In the five combination therapy regimens, mice were randomly divided into 4 groups, namely: normal saline control and adeno-associated virus vector interfering with CMTM6 expression treatment group (AAV); candidate combination therapy treatment group (PD-L1 antibody or imiquimod or doxorubicin or fluvastatin or metformin); and adeno-associated virus vector interfering with CMTM6 expression combined with drugs (AAV+combined drugs).

The dosages of AAV administration were all 1×10¹⁰ vg; PD-L1 antibody was 100 μg/mouse, administered three times, once every three days; imiquimod was 50 μg/mouse, administered five times, once every three days; metformin was 200 μg/mouse, administered five times, once every two days; doxorubicin was 100 μg/mouse, administered three times, once every three days; and fluvastatin was 200 μg/mouse, administered five times, once every two days.

The body weight of the mice as well as the length (L) and width (W) of the tumors were observed and recorded until the endpoint of dissection. The tumor volume (V) was calculated using the formula V=(L×W×W)/2, the tumor growth curves were plotted, and the tumor inhibition rates were calculated.

After the subcutaneous tumors of the mice had grown to a certain length, euthanasia was performed and the animals were dissected to take out the subcutaneous tumors. The tumor growth curves and endpoint tumor weights of mice were recorded.

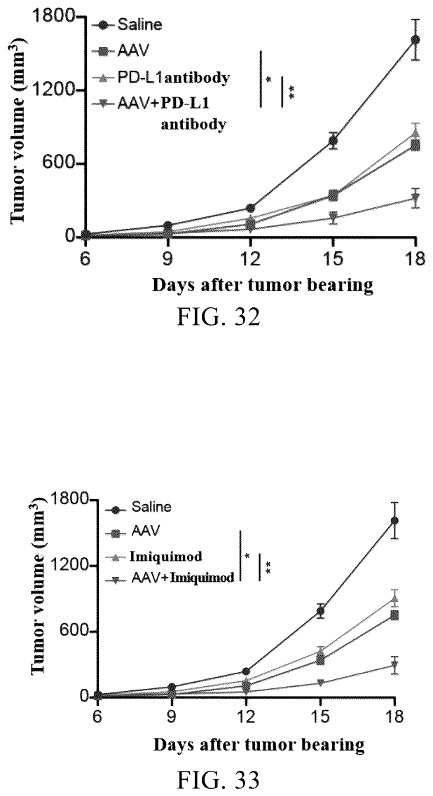

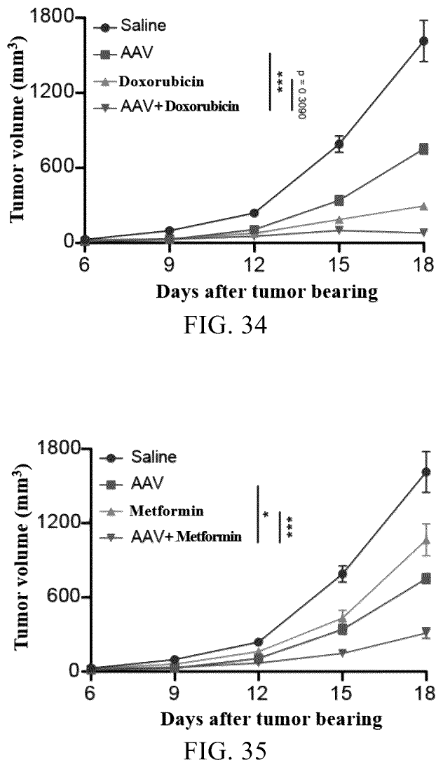

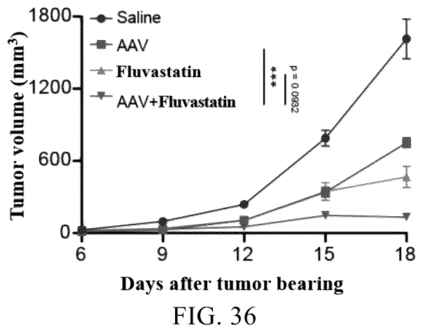

As shown in FIGS. 32, 33, 34, 35 and 36, the results showed that in the CT26 colorectal cancer xenograft model, the adeno-associated virus for interfering with CMTM6 expression of the present invention could be widely used in combination with other drugs to achieve better anti-tumor effects. The adeno-associated virus for interfering with CMTM6 expression of the present invention could effectively improve the anti-tumor effects when combined with immune checkpoint antibodies, immune agonists, chemotherapy drugs, lipid metabolism regulating drugs, and glucose metabolism regulating drugs, and the anti-tumor effect of the combination therapy could reach a maximum inhibition rate of 95.02%. Among them, the combination with chemotherapy drugs and lipid metabolism regulating drugs has better tumor inhibition effects.

### Example 13. Evaluation of the Effect of Adeno-Associated Virus Vectors Interfering with CMTM6 Expression on the Tumor Immune Microenvironment

At the end point of the mouse dissection of the CT26 xenograft tumor model in Example 10, the subcutaneous tumors were taken out, 150 mg of tumor tissue was cut into a meat puree and digested at 180 rpm at 37° C. in hyaluronidase and collagenase IV for 1.5 h, and then processed into a single-cell suspension. The pretreated cell suspension was divided into two aliquots, one for immunophenotyping of myeloid cells and tumor cells, and one for immunophenotyping of lymphocytes.

Treatment of myeloid cell and tumor cell sample suspension: cells were treated with 2-4 mL sterile red blood cell lysis buffer for 8 minutes and terminated with PBS buffer to remove red blood cells and debris from the sample suspension. The cell samples were washed for later use.

Treatment of lymphocyte sample suspension: cells were centrifuged with lymphocyte separation solution to obtain a lymphocyte isolation layer, i.e., the tumor lymphocyte suspension, which was washed for later use.

Myeloid cell and tumor cell samples and lymphocyte samples were blocked with Fc receptor blocking solution at 4° C. for 1 hour to remove non-specific staining caused by the Fc receptor. Cell surface protein staining was performed, followed by staining on ice for 20 min. Then, intracellular protein staining was performed, and after the cells underwent fixation and membrane permeabilization, the cells were subjected to membrane permeabilization staining and then stained on ice for 20 minutes. Following rinsing, the sample cell suspension was resuspended and analyzed by flow cytometry.

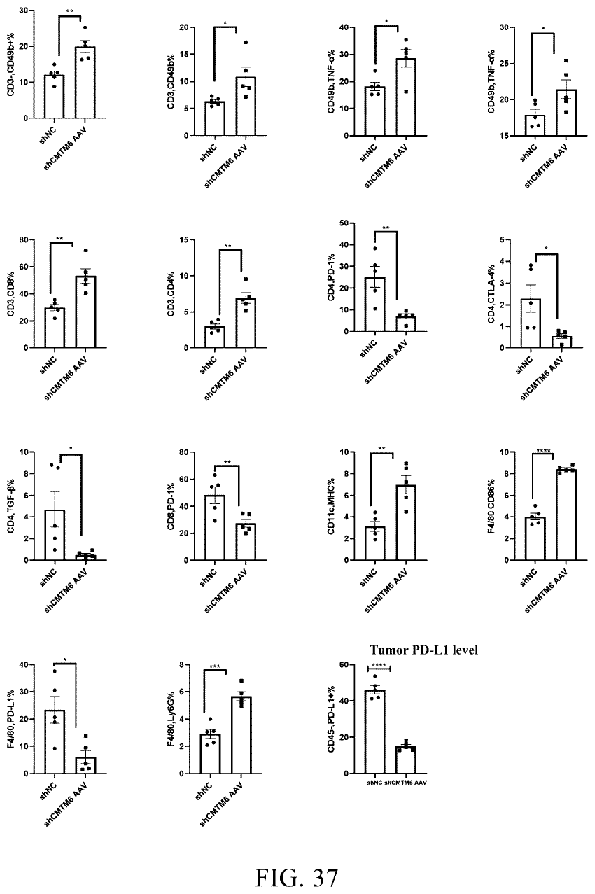

Flow cytometry immunophenotyping results were shown in FIG. 37. The adeno-associated virus for interfering with CMTM6 expression of the present invention targeted the tumor immune microenvironment, could reshape the tumor immune microenvironment, and coordinate innate immunity and adaptive immunity to exert anti-tumor activity. In particular, the activity of cytotoxic cells, such as CD8+ T cells and NK cells, was mobilized, which could promote their secretion of powerful anti-tumor factors TNF-α and granzyme, etc. The AAV of the present invention could also reduce the immunosuppressive analysis of CD4+ T cell expression, such as PD-1, CTLA-4, etc. At the same time, the AAV of the present invention also reduced the PD-L1 expressed by tumor cells.

In summary, the adeno-associated virus for interfering with CMTM6 expression of the present invention can achieve anti-tumor growth effects by targeting tumor tissues, regulating the tumor immune environment, mobilizing anti-tumor immune responses, and downregulating inhibitory tumor immunity.

### Example 14. Construction of Adeno-Associated Viruses Interfering with CMTM6 Expression with RGD Polypeptide Modification

The optimal administration mode of the adeno-associated virus constructed and applied in the above examples of the present invention was peritumoral administration, which contributed to improving its anti-tumor effects. Although the technology of orthotopic administration of tumors is mature, it is relatively inconvenient than intravenous injection and other systematic administration methods, and patient compliance is not high.

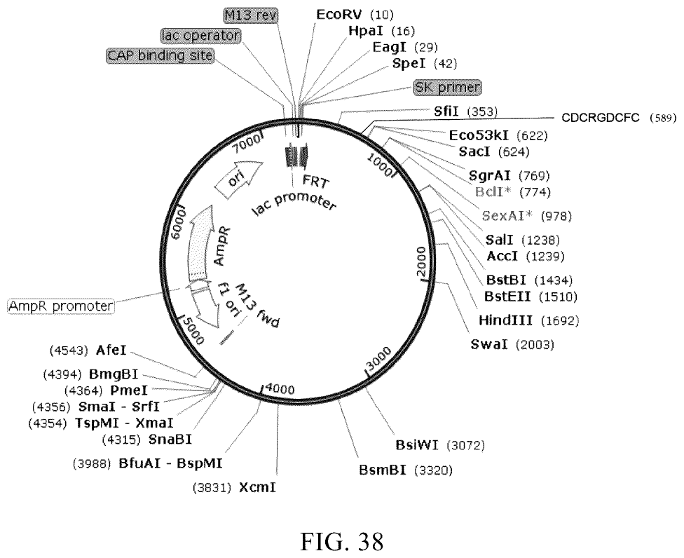

Therefore, the inventors constructed an RGD polypeptide-modified adeno-associated virus that interferes with CMTM6 expression. The overall construction method was consistent with the construction method of AAV genome vector in Example 7, except that the viral capsid needed to be modified by inserting an amino acid sequence (represented by CDCRGDCFC) at position 589 of the virus packaging plasmid pAAV-RC9, as shown in FIG. 38.

In this way, the AAV capsid packaged with pAAV-RC9-RGD has an RGD polypeptide sequence on it, which can specifically target tumor cells with high expression of integrins and can be used for systemic drug delivery.

### Example 15. Evaluation of In Vivo Anti-Tumor Activity of Adeno-Associated Virus Interfering with CMTM6 Expression with RGD Polypeptide Modification

In order to evaluate the systematic anti-tumor activity of the RGD polypeptide-modified adeno-associated virus interfering with CMTM6 expression constructed in Example 14 of the present invention, the inventors adopted the CT26 xenograft model for evaluation.

Resuscitate CT26 colorectal cancer tumor cells and passage them to ensure that the tumor cells have been passaged for at least 3 generations at the time of tumor bearing. CT26 tumor cells were inoculated subcutaneously into female BALB/c mice, and the inoculation dose was 5×10⁵ cells/mouse. The mice were randomly divided into 2 groups, namely: control shRNA adeno-associated virus treatment group (shNC AAV-RGD) and RGD polypeptide-modified adeno-associated virus vector interfering with CMTM6 expression treatment group (shCMTM6 AAV-RGD).

The administration dosages were all 1×10¹⁰ vg, intraperitoneal injection. The endpoint volumes and endpoint weights of mice were recorded and statistically analyzed.

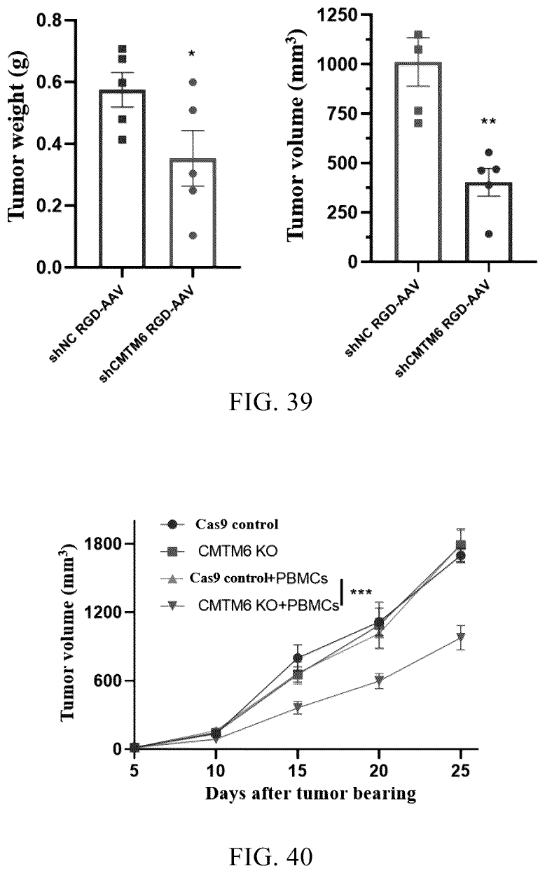

As shown in FIG. 39, the results showed that in vivo, the RGD polypeptide-modified adeno-associated virus vector interfering with CMTM6 expression of the present invention could still significantly inhibit the tumor growth of CT26 colorectal cancer through systematic administration, expanding the convenience of the application of the gene therapy method used in the present invention.

### Example 16. Evaluation of the Anti-Tumor Activity of Lentivirus Vectors for Knocking Out CMTM6 Expression in Immune Humanized Systems

In the present example, the inventors carried out an anti-tumor activity evaluation experiment on human-derived RKO colon cancer tumors in an immune humanized system.

Female NSG mice were randomly divided into 4 groups, namely (1) Cas9 control group, (2) CMTM6 KO group, (3) Cas9 control+PBMCs group, and (4) CMTM6 KO+PBMCs group. Three days prior to tumor bearing, four groups of mice were treated with pet electric clippers to remove hair under the axilla of the right forelimb and the surrounding area.

For groups (1) and (2): human colorectal cancer tumor RKO cells were cultured for at least 3 generations after cell resuscitation. When the cells were in good condition and in the logarithmic growth phase, the tumor cells were digested with trypsin solution containing 0.05% EDTA and centrifuged, washed twice with cold PBS, and then resuspended with cold PBS and counted;

For groups (3) and (4): PBMC cells isolated from the same person were resuscitated and used immediately. PBMCs were mixed with RKO cells to achieve a RKO density of 5×10⁷ cells/mL and a PBMC density of 1×10⁷ cells/mL.

Insulin needles were used to inoculate 100 μL of the above cells subcutaneously in the axilla of the right forelimb of the four groups of NSG mice, and the cells were kept on ice.

The inoculation dose/tumor load of groups (1) and (2) was 5×10⁶ cells; the inoculation dose/tumor load of groups (3) and (4) was 5×10⁶ RKO cells and 1×10⁶ PBMC cells.

After tumor bearing, the tumor volumes of mice were monitored every 5 days for a total of 5 time points.

The experimental results were shown in FIG. 40, indicating that the knockout of CMTM6 significantly inhibited the in vivo growth of human RKO tumors in a humanized tumor model of RKO/human PBMCs mixed tumor-bearing.

Therefore, the lentivirus vector for knocking out CMTM6 expression of the present invention also has significant inhibitory effects on human-derived tumors.

### DISCUSSION

Tumor immunotherapy has achieved breakthroughs in the field of cancer treatment, yet it still faces challenges such as suboptimal efficacy and low response rates, etc., that necessitate resolution. Taking PD-1/PD-L1 immune checkpoint antibody therapy as an example, there are numerous tumor types that do not respond to this treatment in clinical practice, and in some tumor indications, it may even lead to hyperprogression of tumors, significantly limiting the application of these antibody drugs. On the other hand, to enhance the response rate of PD-1/PD-L1 antibodies, the discovery and application of therapeutic biomarkers have emerged as a major research direction. Currently, factors like PD-L1 expression levels and tumor mutation burden are used to select patients for administration, but the issue of medication for patients who do not meet the criteria still needs to be solved.

Exploring novel targets may be one of effective approaches to address the aforementioned issues. When applying these new targets, it is crucial to address the pain points of PD-1/PD-L1 antibodies, aiming to benefit patients with low PD-L1 expression, cold tumors, or resistance to PD-1/PD-L1 antibody therapy, either through monotherapy or combination therapy.

The present invention provides for the first time a gene therapy vector that interferes with CMTM6 expression, along with modifications and multi-type combination therapies regarding this gene therapy vector. The gene therapy vector of the present invention targets the novel anti-tumor target CMTM6, regulating tumor cell growth in vivo by interfering with CMTM6 gene expression. It inhibits the in vivo growth of colorectal cancer, melanoma, breast cancer, non-small cell lung cancer, liver cancer, etc., and demonstrates remarkable efficacy against tumors with low or no PD-L1 expression, as well as those resistant to PD-1/PD-L1/CTLA-4 antibodies. Furthermore, the gene therapy vector that interferes with CMTM6 expression potently stimulates intra-tumoral anti-tumor immune responses, notably activating CD8⁺ T cells and NK cells, underscoring the potential of this target as a novel target for tumor immunotherapy.

All literatures mentioned in the present invention are incorporated herein by reference, as though each one is individually incorporated by reference. In addition, it should be understood that, after reading the above teachings of the present invention, those skilled in the art can make various changes or modifications to the present invention, these equivalents also fall within the scope as defined in the appended claims of the present application.

1. Use of a gene therapy vector for targeted downregulation of CMTM6, in the manufacture of a composition or formulation, wherein the composition or formulation is used for: (a) prevention and/or treatment of tumors; and/or (b) inhibition of tumor cells.

2. The use of claim 1, wherein the gene therapy vector is selected from the group consisting of:
(Z1) a lentivirus for targeted inhibition of CMTM6 expression;
(Z2) an adeno-associated virus for targeted inhibition of CMTM6 expression;
(Z3) a lentivirus for simultaneous targeted inhibition of CMTM6 expression and PD-L1 expression;
(Z4) an adeno-associated virus for simultaneous targeted inhibition of CMTM6 expression and PD-L1 expression;
(Z5) any combinations of Z1˜Z4 mentioned above.

2. The use of claim 1, wherein the gene therapy vector is used in combination with a drug selected from the group consisting of: an immune checkpoint antibody, an immune agonist, a chemotherapy drug, a lipid metabolism regulating drug, a glucose metabolism regulating drug, an additional gene therapy vector, and a combination thereof.

4. The use of claim 1, wherein the tumors are those that have shown ineffectiveness or failure for treatment with immune checkpoint antibodies or immune checkpoint inhibitors, or those that are unsuitable for treatment with immune checkpoint antibodies or immune checkpoint inhibitors; preferably the tumors are tumors with low or no expression of PD-L1.

5. A viral or non-viral vector capable of targeted inhibition of CMTM6 expression in tumors and/or cells, which carries or contains a coding sequence for inhibiting CMTM6 expression.

6. The vector of claim 5, wherein the coding sequence for inhibiting CMTM6 expression is a sgRNA or shRNA for targeted inhibition of CMTM6, comprising:
(i) one or more selected from SEQ ID NOs: 1-12 or derived sequences thereof; or
(ii) one or more selected from SEQ ID NOs: 13-15 or derived sequences thereof.

7. A dual-targeting viral vector capable of simultaneous targeted inhibition of CMTM6 expression and PD-L1 expression in tumors and/or cells, wherein a coding sequence carried or contained in the dual-targeting viral vector is selected from the group consisting of:
(i) one or two selected from SEQ ID NOs: 1-15 or derived sequences thereof; and
(ii) one or two selected from SEQ ID NOs: 16-20 or derived sequences thereof.

8. A polynucleotide encoding the genome of a vector selected from the group consisting of:
(1) a viral or non-viral vector capable of targeted inhibition of CMTM6 expression in tumors and/or cells, which carries or contains a coding sequence for inhibiting CMTM6 expression; or
(2) the dual-targeting viral vector of claim 7.

9. An expression vector comprising the polynucleotide of claim 8.

10. A host cell comprising the expression vector of claim 9.

11. A nucleic acid conjugate, comprising:
(a) the polynucleotide of claim 8; and
(b) other coupled moiety selected from the group consisting of: small molecule compounds, PEG, fluorescein, radioactive isotopes, fatty acid chains, protein fragments, polypeptides, and combinations thereof.

12. A pharmaceutical formulation, comprising:
(a) the expression vector of claim 9; and
(b) a pharmaceutically acceptable carrier.

13. A method for preventing and/or treating tumors, comprising the step of: administering the expression vector of claim 9, or a pharmaceutical formulation comprising: (a) the expression vector of claim 9; and (b) a pharmaceutically acceptable carrier, to a subject in need thereof.
